(19) United States
(12) Patent Application Publication
(10) Pub. No.: US 2009/0214496 A1
(43) Pub. Date: Aug. 27, 2009

(54) BMX/ETK TYROSINE KINASE GENE THERAPY MATERIALS AND METHODS

(75) Inventors: Kari Alitalo, Helsinki (FI); Wang Min, New Haven, CT (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignees: LICENTIA LTD., Helsinki (FI); YALE UNIVERSITY, New Haven, CT (US)

(21) Appl. No.: 12/162,511

(22) PCT Filed: Jan. 30, 2007
(86) PCT No.: PCT/US07/02537

§ 371 Date: Jan. 12, 2009

Related U.S. Application Data
(60) Provisional application No. 60/764,123, filed on Jan. 30, 2006.

(51) Int. Cl. A61K 35/12; A61K 31/7088; A61K 38/02; C07H 21/00

(52) U.S. Cl. 424/93.21; 514/44 R; 514/2; 536/23.2

(57) ABSTRACT

The present invention relates to materials and methods for the treatment of arterial diseases having impaired arteriogenesis and angiogenesis. More particularly, the invention provides materials and methods for treating arterial disease using a gene therapy vector expressing Bmx tyrosine kinase.

The present application claims the priority benefit of U.S. Provisional Patent Application No. 60/764,123, filed Jan. 30, 2006, hereby incorporated by reference.

This invention was made with government support under grant number R01HL65978-5 awarded by the National Institutes of Health. The government has certain rights in the invention.

### FIELD OF THE INVENTION

The present invention relates to materials and methods for promoting arteriogenesis and angiogenesis in patients having impaired arteriogenesis and poor vasculature.

### BACKGROUND OF THE INVENTION

Angiogenesis is the sprouting of new capillaries from existing vasculature. Angiogenesis is initiated by endothelial cell migration and proliferation and results in remodeling of the extracellular matrix and expansion of the surrounding vascular tissues. (Collison and Donnelly, Eur. J. Endovasc. Surg. (2004) 28:9-23). Vasculogenesis, the process of blood vessel formation occurring by a de novo production of endothelial cells, requires migration of bone marrow-derived endothelial precursor cells which differentiate into endothelial cells and fuse to luminal surfaces (Collison and Donnelly, supra).

Arteriogenesis is an increase in the size of pre-existing arteriolar collateral connections by recruitment of perivascular cells and expansion and remodeling of the extracellular matrix (Ito et al., Am J. Physiol. (1997) 273:H1255-65). As a result of shear stress, arteriogenesis increases the size and wall thickness of collateral vessels (Collison and Donnelly, supra), resulting in improved blood flow through pre-formed collateral arterioles. Collaterals are often capable of fully supplementing blood flow to the myocardium distal to a coronary occlusion, preserving myocardial function.

The vascular endothelium controls important properties, such as inflammatory responses, the regulation of a nonthrombogenic (non-clot-forming) surface and responses of the vessel wall to products of platelet activation on the vessel wall, and recanalization of vascular occlusions by thrombi or blood clots. Arteriosclerosis is considered to arise in part because of failure of the nonthrombogenic vessel surface. Characteristic locations for arteriosclerosis are sites of arterial bifurcations and sites of high shear pressure and turbulent blood flow. At such sites, microaggregates of platelets are thought to form and be activated, resulting in the release of substances that promote arteriosclerosis.

The vascular endothelium of arteries is subject to physiological stresses, such as high intravascular pulsatile pressure and shear stress, which have been shown to control gene expression in endothelial cells (Gimbrone et al. J. Clin. Invest. (1997) 100 (11 Suppl):S61-5). Such influences on the endothelial cells, including readjustment of gene expression to stressful conditions, are controlled by signals from the extracellular fluid and pericellular matrix via specific cell surface receptors, such as hemopoietin-cytokine receptors, receptor tyrosine kinases and G-protein coupled receptors (Guidebook to Cytokines and Their Receptors, Nicos A. Nicola (Ed.), Oxford University Press, 1994; herein incorporated by reference). For example, shear stress has been shown to lead to changes in the TGFβ-SMAD signal transduction in endothelial cells, which is considered, among other things, to lead to changes in the biosynthesis of extracellular matrix components by the cells (Topper et al. Proc. Natl. Acad. Sci. USA (1997) 94:9314-19).

Tyrosine protein kinases (TKs) are essential components of signal transduction in endothelial cells. Several of the TKs function as transmembrane receptors, transducing signals from growth factors to the cytoplasm (Mustonen and Alitalo, J. Cell Biol. (1995) 129: 895-98). The extracellular domains of the receptor TKs are responsible for ligand binding, while the intracellular TK domains transmit the activation signals through phosphorylation of cellular polypeptides. Five different endothelial cell receptor TKs are known, encoded by two different gene families (Mustonen and Alitalo, supra). Non-receptor tyrosine kinases relatively specific for endothelial cells also have been reported (Vihinen and Smith, Crit. Rev. Immunol. (1996) 16(3):251-75).

Bmx tyrosine kinase (bone marrow tyrosine kinase in chromosome-X), also named Etk, (endothelial/epithelial tyrosine kinase), is a member of Tec family non-receptor tyrosine kinases. Bmx and three other members of this family, (Btk, Itk and Tec), participate in signal transduction in response to growth factor receptors, cytokine receptors, G-protein coupled receptors, antigen receptors, and integrins [Tamagnone et al., Oncogene (1994) 9:3683-88; Qiu, et al., Proc Natl Acad Sci USA (1998) 95:3644-3649.; Chau et al., Oncogene (2002) 21:8817-8829; Tsai et al., Mol Cell Biol (2000) 20:2043-2054; Mao et al., Embo J (1998) 17:5638-5646; Chen et al., Nat Cell Biol (2001) 3:439-444]. The four proteins encoded by members of this Btk gene family share substantial homology, including typical Src homology (SH) SH2 and SH3 domains upstream of the TK domain. A special feature of these TKs is a pleckstrin homology (PH) domain in the N-terminal region (Musacchio et al. TIBS (1993) 18:343-48) and a TEC homology (TH) domain which has a PXXP motif (with the exception of Bmx).

Several of these non-receptor TKs have been shown to be expressed in various cultured hematopoietic cell lines. The Tec TK is expressed in all murine hematopoietic cell lines examined according to Mano et al., Oncogene (1990) 5:1781-86. The Tec kinase is activated by multiple cytokine receptors in the hematopoietic cells and by thrombin and integrin signals in blood platelets (Hamazaki et al., Oncogene (1998) 16:2773-79). Itk (Gibson et al. Blood (1993) 82:1561-72) and Btk (de Weers et al. Eur J Immunol. (1993) 23:3109-14) are selectively expressed at certain stages of lymphocyte development and the expression of the Txk TK has been assigned to T-cells (Sommers et al., Oncogene (1995) 11:245-51).

Bmx is highly expressed in cells with great migratory potential including metastatic tumor cells and endothelial cells (EC) [Chen et al., Nat Cell Biol (2001) 3:439-444; Ekman et al., Circulation (1997) 96:1729-32; Bagheri-Yarmand et al., J Biol Chem (2001) 276:29403-09]. Bmx can be activated by various angiogenic stimuli such as integrin engagement via focal adhesion kinase (FAK) (Chen et al, supra), by VEGF stimulation via VEGFR-1, Tie-2 [Rajantie et al., Mol Cell Biol. (2001) 21:4647-55] or VEGFR-2 [Chau et al., Oncogene (2002) 21:8817-8829], by TNF stimulation via TNFR2 [Pan et al., Mol Cell Biol (2002) 22:7512-23] or/and via transactivation by VEGFR-2 [Zhang et al., J Biol Chem (2003) 278:51267-76]. However, the mechanism by which Bmx mediates EC migration has not been determined. Several downstream effectors of Bmx involved in cell migration have been reported. Bmx, through its PH domain, directly binds to and activates Rho A (but not Rac1 and Cdc42) [Kim et al., J Biol Chem (2002) 277:30066-71]. Similarly, Bmx, through its PH domain, binds to and activates PAK1 (Bagheri-Yarmand et al., supra), a 65-kDa serine/threonine kinase implicated in integrin-induced EC migration and angiogenesis by modulating EC contraction [Kiosses et al., Circ Res (2002) 90:697-702]. It has also been shown that Bmx mediates the TNF-induced PI3K-Akt angiogenic pathway (Zhang et al., supra) which has been well documented in growth factor-stimulated cell migration [Qi et al., Exp Cell Res (2001) 263:173-182.; Gille et al., EMBO J (2000) 19:40664-73; Dimmeler et al., FEBS Lett (2000) 477:258-262; Kureishi et al., Nat Med (2000) 6:1004-10; Ackah et al., J Clin Invest (2005) 115:2119-27].

However, the role of Bmx in vivo is not clear. It has been shown that the Bmx-deficient mice are normal in embryonic angiogenesis (Rajantie et al., supra). Recent data using mice having the Bmx-transgene specifically expressed in epidermal keratinocytes suggest that Bmx may be involved in wound reepithelialization [Paavonen et al. Mol Biol Cell (2004) 15:4226-4233]. Although Bmx is primarily expressed in bone marrow and arterial endothelium in vivo, the roles of Bmx in inflammatory arteriogenesis and angiogenesis are not known.

Peripheral arterial disease (PAD) is a common disease found in 10-25% of patients over the age of 55 years old and its occurrence and severity are strongly correlated with other cardiovascular risk factors that lead to coronary artery disease and stroke, such as hyperlipidemia, smoking and endothelial dysfunction. PAD frequently occurs secondary to atherosclerosis and/or diabetes resulting in narrowing of the arteries in the legs, thus limiting blood flow to the extremities. An animal model of PAD has been created by ischemia injury of mouse hindlimb. Mechanistically, ischemia-induced vascular remodeling occurring in both human patients and animal models can be explained as follows: stenotic narrowing of the common ileac, femoral artery or branch off the femoral artery leads to tissue ischemia, thereby creating regional hypoxia and mismatch of the oxygen supply versus demand for oxygen in skeletal myocytes or myotubes. The reduction in tissue perfusion correlates with activation of vascular endothelial cells and the recruitment of inflammatory cells, in particular macrophages. Activated macrophages then secrete chemokines and cytokines, such as vascular endothelial growth factor (VEGF), and tumor necrosis factor (TNF), that may participate in arterializing pre-formed collaterals (arteriogenesis) to provide a stable channel for blood flow to the distal limb.

Contemporaneously, redistribution of blood flow and the attendant changes in shear stress may synergize with local cytokines to arterialize immature collaterals and perhaps induce angiogenesis. Once a stable, collateral circulation has been established, the improvement in distal blood flow and shear stress triggers an increase in capillary angiogenesis, thus increasing capillary to fiber ratios and oxygen delivery to the dependent portions of the lower limb. Therefore, proportionally regulated arteriogenesis and angiogenesis are necessary to improve nutritive blood flow to tissue and promote functional limb salvage after injury.

There remains a need in the art to regulate and improve arteriogenesis and angiogenesis in ischemic tissue and after ischemic injury and speed blood flow to sites affected by injury to the vascular endothelium. Agents to improve angiogenesis and arteriogenesis may increase and speed recovery of blood flow to sites of ischemia or injury, thereby resulting in less damage to patients caused by lack of blood and oxygen to distal parts of the body.

### SUMMARY OF INVENTION

The invention provides materials and methods that are useful for prophylaxis and therapy for circulatory disorders.

For example, in one embodiment, the invention is a method for increasing arteriogenesis or angiogenesis in a mammalian subject. Such a method is useful for prophylaxis and therapy for any disorders characterized by inadequate circulation, such as atherosclerosis, PAOD, and other disorders.

In one variation, the method comprises: administering to a mammalian subject in need of arteriogenesis or angiogenesis a composition that comprises a polynucleotide that comprises a nucleotide sequence that encodes a Bmx tyrosine kinase amino acid sequence. In some variations of this method, a native or “wildtype” Bmx amino acid and/or encoding nucleic acid sequence is utilized. In preferred variations, the native sequence is from the same species as the species to be treated, .e.g., a human Bmx sequence is introduced into a human subject. In other variations, species homologs (orthologs) are used. In still other variations, non-naturally occurring sequences with Bmx tyrosine kinase activity are used.

For example, in some embodiments, the Bmx tyrosine kinase amino acid sequence is selected from the group consisting of: (a) the amino acid sequence set forth in SEQ ID NO: 2; (b) the amino acid sequence set forth in SEQ ID NO: 4; (c) fragments of (a) or (b) that retain Bmx tyrosine kinase activity; and (d) amino acid sequences that are at least 70% identical to (a), (b), or (c) and that retain Bmx tyrosine kinase activity. With respect to sequence similarity, greater similarity to SEQ ID NOs: 2 or 4 (or fragments) is preferred, e.g., at least 75%, 80%, 85%, 90%, 91%, 92%, 93%, 94%, 95%, 96%, 97%, 98%, 99%, or 99.5% identity to SEQ ID NO: 2 or 4 or fragments thereof.

As described below in greater detail, SEQ ID NO: 2 comprises a human Bmx amino acid sequence, and SEQ ID NO: 4 comprises a constitutively active variant thereof. Domain analysis of the Bmx protein as determined on the Swiss-Prot database (Swiss-Prot Accession No. P51813) indicates that the pleckstrin homology (PH) domain is found from approximately residues 4-111 of SEQ ID NO: 2, while the SH2 domain is located from approximately residues 296-392 of SEQ ID NO: 2, and the Bmx protein kinase domain is located at approximately residues 417 to 675 of SEQ ID NO: 2. The SH3 domain has been determined to lie at approximately residues 213-265 of SEQ ID NO: 2.

Bmx variants that contain mutations in the kinase domain (e.g., K444Q) are more likely to diminish or destroy kinase activity, and such kinase-destroying mutants are to be avoided. An immunocomplex kinase assay such as described in Rajantie et al., “Bmx tyrosine kinase has a redundant function downstream of angiopoietin and vascular endothelial growth factor receptors in arterial endothelium,” Mol Cell Biol., 2001 21:4647-55, incorporated herein by reference, are suitable for confirming that a Bmx variant retains kinase activity. Also, mutations in the PH domain (e.g., E42K) that diminish membrane association should be avoided.

Additional guidance for selecting amino acids suitable for modification or removal are provided below. Generally speaking, conservative amino acid changes are less likely to diminish wildtype Bmx tyrosine kinase activity, and are thus a preferred class of changes. Alignment of species homologs, to identify which residues evolution has shown to be more amenable to variation and which residues are more highly conserved, also provides guidance as to residues that are suitable for modification (as well as some of the suitable choices for substitution).

In some embodiments of the invention, a Bmx trangene is introduced into cells ex vivo, and thereafter, the cells are administered to a subject in need of therapy.

For example, in one embodiment, the invention is a method for increasing arteriogenesis or angiogenesis in a mammalian subject comprising: administering to a mammalian subject in need of arteriogenesis or angiogenesis a composition that comprises cells transduced with a polynucleotide that causes elevated expression of a Bmx tyrosine kinase amino acid sequence. As already explained, in some variations, the Bmx tyrosine kinase amino acid sequence is selected from the group consisting of: (a) the amino acid sequence set forth in SEQ ID NO: 2; (b) the amino acid sequence set forth in SEQ ID NO: 4; (c) fragments of (a) or (b) that retain Bmx tyrosine kinase activity; and (d) amino acid sequences that are at least 70% identical to (a), (b), or (c) and that retain Bmx tyrosine kinase activity.

In some variations, the polynucleotide is an expression control sequence or a polynucleotide that encodes a protein other than Bmx, whose expression leads to elevated Bmx expression. In other variations, the polynucleotide encodes the Bmx amino acid sequence, and the expression of the polypeptide in the cells directly results in elevated expression of Bmx tyrosine kinase.

In some variations of the ex vivo gene therapy, the cells are selected from the group consisting of endothelial cells, endothelial precursor cells, and bone marrow derived cells such as monocytes, macrophages, and stem cells with the potential to differentiate into endothelial cells.

For both in vivo and ex vivo embodiments of the invention, the polynucleotide preferably includes suitable expression control sequences to assure that it is properly expressed in the target host cells. In some variations, “naked” DNA or RNA gene therapy is contemplated, in which polynucleotides are injected directly into target tissue or cell culture. In some variations, the composition comprises a vector that contains a transgene that includes the polynucleotide, operably linked to at least one expression control sequence that promotes expression of the polynucleotide in mammalian cells. Exemplary expression control sequences include promoters, enhancers, introns, 3′UTR sequences, zinc-finger constructs, and polyadenylation signal sequences.

Any expression control sequence that improves expression in target cells may be used in variations of the invention. In some variations, the promoter is cell-type specific and/or is induced by certain physiological states, e.g., hypoxia. In other variations, a constitutively active promoter is used. Exemplary promoter sequences include: a CMV promoter, a M-actin promoter, a Tie promoter, a Tie-2 promoter, a VE-cadherin promoter, an endothelial cell specific promoter, a bone-marrow specific promoter, and an inducible Tet promoter

Exemplary vectors include adenoviral vectors, an adeno-associated viral vectors, lentivirus vectors, plasmids, and liposomes. With respect to viral vectors, replication-deficient forms are preferred in many variations of the invention.

In methods of the invention, any route of administration may be used to deliver the polynucleotide (or vector) or transduced cell composition to the subject in need of treatment. In some variations, the administration is systemic, e.g., via intravenous administration into the circulatory system. In other variations, the composition is administered to the subject locally to a site in need of arteriogenesis or angiogenesis. For example, the composition can be administered via a catheter, a membrane, a collar, or a syringe to a site in the body that would benefit from angiogenesis or arteriogenesis to improve circulation, such as a sclerotic vessel or neighboring muscle tissue. In some variations, the composition is administered in combination with a drug-eluting stent. Stents and catheters represent exemplary methods for administration of the composition to a lumen wall of a blood vessel.

Methods of the invention can be used to treat a subject suffering from any disease or condition associated with impaired circulation. For example, in some embodiments, the disease or condition is selected from the group consisting of coronary artery disease, peripheral arterial disease, ischemic injury, arterial stenosis, cerebrovascular disease, and renal arterial stenosis. Exemplary conditions characterized by arterial stenosis include atherosclerosis, stenosis of the heart and leg muscles, and stenosis of diabetic patients.

In preferred variations of the invention, the composition to be administered further includes a pharmaceutically acceptable carrier. Selection of suitable carriers is within the skill in the field of the invention, depending on the active agents in the composition (naked DNA, liposomes, viral vectors, whole cells, etc.); the route of administration, patient, dose, etc.

The Bmx gene therapy of the invention, in vivo or ex vivo, can be practiced alone or in combination with additional therapeutic agents or second agents. In some variations, the additional therapeutic agents enhance the expression of Bmx or the beneficial effects of BMX expression. In other variations, additional therapeutic agents have angiogenic or arteriogenic activity that complements the activity of the BMX therapy, preferably in a synergistic way. In still other variations, the additional therapeutic agents are for the purpose of reducing symptoms and side effects of the disease or condition or the BMX therapy.

For example, in one variation of the invention, the composition further includes, or is co-administered with, a cell-permeable peptide, such as the third alpha-helix of the Antennapedia (Antp) homeodomain or fragment of HIV Tat protein, that can cross the cell membrane through a receptor-independent mechanism. See, e.g., Schwarze et al., “In vivo protein transduction: delivery of a biologically active protein into the mouse.” Science 285, 1569-1572 (1999); Derossi, D. et al., Trojan peptides: the penetration system for intracellular delivery. Trends Cell Biol. 8, 84-87 (1998), all incorporated herein by reference.

In some variations, the method of the invention further includes a monitoring step to evaluate the efficacy of therapy. For example, measuring changes in flow, or imaging circulation in an ischemic tissue, or measuring the ability of the subject to perform in an exercise or stress test indicate whether to repeat the administration of the composition to further increase angiogenesis or arteriogenesis; or indicate to cease the administration when a desirable improvement has been measured.

In a related embodiment, the polynucleotides, vectors, transduced cells, and compositions comprising these agents are themselves an aspect of the invention. Likewise, combinations of any of the foregoing with each other, or with co-therapy agents described herein, are another aspect of the invention. Similarly, medical devices such as catheters, collars, or stents, that are modified to contain compositions of the invention for therapeutic delivery, are another aspect of the invention. For example, a catheter such as a balloon catheter that has avoid in which a composition of the invention is placed is an aspect of the invention. A drug-coated stent, wherein the “drug” comprises a composition described herein, is another example of a device of the invention.

In a related embodiment, the invention includes use of polynucleotides, vectors, transduced cells, and medical devices for prophylaxis or therapy. For example, the invention includes the use of a polynucleotide that encodes a polypeptide with Bmx tyrosine kinase activity for the manufacture of a medicament to treat coronary artery disease or peripheral artery disease. Exemplary polypeptides are described throughout the summary and detailed description, and include polypeptides that comprise an amino acid sequence selected from the group consisting of: (a) the amino acid sequence set forth in SEQ ID NO: 2; (b) the amino acid sequence set furth in SEQ ID NO: 4; (c) fragments of (a) or (b) that retain BMX tyrosine kinase activity; and (d) amino acid sequences that are at least 70% identical to (a), (b), or (c) and that retain BMX tyrosine kinase activity.

To provide another example, the invention includes use of transduced cells for the manufacture of a medicament for prophylaxis or therapy to treat coronary artery disease or peripheral artery disease, wherein the cells are transformed or transfected with a polynucleotide that encodes a polypeptide with BMX tyrosine kinase activity. Exemplary polypeptides are described throughout the summary and detailed description.

Additional features and variations of the invention will be apparent to those skilled in the art from the entirety of this application, including any drawing and the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION OF THE INVENTION

The present invention addresses a problem in the art of regulating and improving arteriogenesis and angiogenesis in ischemic tissue and after ischemic injury, and speeding blood flow to sites affected by ischemia or injury to the vascular endothelium. The invention provides materials and methods for increasing arteriogenesis and angiogenesis in a mammalian subject comprising administering gene therapy to the subject.

As used herein, “increasing arteriogenesis and angiogenesis” in a subject refers to a detectable improvement in any one of arterial size, wall thickness, increased blood flow, increased number of collateral arteries in a subject, and enhanced recovery from ischemic injury. Evidence of an increase can be direct (measurement of vessels or blood flow, for example), or indirect (e.g., measurement of improved muscle tone or contractile force, etc.).

As used herein, “Bmx tyrosine kinase” refers to mammalian Bmx tyrosine kinase amino acid sequence, for example the human BMX kinase set out in SEQ ID NO: 2, orthologs of said sequences (described elsewhere herein), fragments of SEQ ID NO: 2 that retain BMX kinase activity and amino acid sequences that are at least 70% identical to SEQ ID NO: 2 and retain Bmx kinase activity. Bmx tyrosine kinase activity refers to the ability of Bmx kinase to stimulate downstream signaling events in endothelial cells as described herein and in the art. For example, a cell or molecular based assay for Bmx activity can readily detect Bmx activity of the native protein, or fragments of variants thereof by western blot with phospho-specific antibody pY40) [Zhang et al., J Biol Chem (2003) 278:51267-76.] Additionally, an immunocomplex assay to detect kinase activity is used to measure Bmx kinase activity [Rajantie, et al, (supra) and Ekman, et al., [Oncogene (2000) 19:4151-4158].

As used herein, “gene transfer” refers to the introduction (in vivo or in vitro) of a desired natural, synthetic, or recombinant gene or gene fragment into a target cell in such a manner that the introduced gene functions stably or transiently in the target cell. Gene function includes transcription and/or translation of mRNA into an encoded protein in the cell. The gene or gene fragment to be introduced according to the present invention encompasses DNA, RNA, or any nucleic acid which is a synthetic analog thereof, having a specific sequence. In the present specification, the terms “gene transfer”, “transfection”, and “transfecting” are interchangeably used. Gene transduction may be used when gene introduction occurs into cells in a tissue in vivo.

The term “operably linked” refers to functionally related nucleic acid sequences. For example, a promoter is operably associated or operably linked with a coding sequence if the promoter modulates the transcription of the coding sequence. While operably linked nucleic acid sequences can be contiguous and in the same reading frame, certain genetic elements, e.g. repressor genes, are not contiguously linked to the coding sequence but still control transcription/translation of the coding sequence.

“Treatment” or “treating” refers to either a therapeutic treatment or prophylactic or preventative treatments. A therapeutic treatment may improve at least one symptom of disease in an individual or may delay worsening of a progressive disease in an individual, or prevent onset of additional associated diseases.

A “therapeutically effective dose” or “effective dose” of a Bmx gene therapy vector refers to that amount of the compound sufficient to result in amelioration of one or more symptoms of the disease being treated. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

### The Bmx Gene and Protein

The Bmx TK gene was isolated while screening for novel TK genes expressed in human bone marrow (Tamagnone et al. Oncogene (1994) 9:3683-88). Because the gene was mapped to chromosome X at Xp22.2, it was called Bmx, Bone Marrow tyrosine kinase gene in chromosome X. The X chromosome is the location of at least one known tyrosine kinase gene-linked disease in humans, the X-linked agammaglobulinemia. Several other human mutations are known in genes located in the X-chromosome, which lead to disease in a hemizygous position in males, because of the lack of a second X-chromosome and thus the lack of a healthy allele. The Bmx tyrosine kinase is a non-receptor tyrosine kinase, meaning that the majority of the protein sequence lies intracellularly bound to the internal cell membrane. The polynucleotide and amino acid sequence of human Bmx tyrosine kinase are set out in SEQ ID NO: 1 and 2, respectively. Domain analysis of the Bmx protein as determined on the Swiss-Prot database (Swiaa-Prot Accession No. P51813) indicates that the pleckstrin homology (PH) domain is found from approximately residues 4-111 of SEQ ID NO: 2, while the SH2 domain is located from approximately residues 296-392 of SEQ ID NO: 2, and the Bmx protein kinase domain is located at approximately residues 417 to 675 of SEQ ID NO: 2. The SH3 domain has been determined to lie at approximately residues 213-265 of SEQ ID NO: 2. In comparison with other Btk family members, Bmx lacks the so-called P-X-P motifs but has extra peptides in between the PH and SH3 and SH2 domains. The SH3 sequence does not conform precisely to the described consensus.

The Bmx genes and/or proteins from other species characterized and sequenced such information and other can be found in databases such as Genbank, maintained by the National Library of Medicine and the National Center for Biotechnology Information. For example, a mouse Bmx mRNA and protein are identified by Genbank Accession Nos. NM_{—}009759 and NP_{—}033889, respectively; the predicted sequences of bovine Bmx tyrosine kinase mRNA and protein are set out in Genbank Accession Nos. XM_{—}610012 and XP 610012, respectively, the predicted sequences of canine Bmx kinase mRNA and protein are set out in Genbank Accession Nos. XM_{—}548870 and XP_{—}548870, respectively; the predicted sequences for rat Bmx tyrosine kinase mRNA and protein are set out in Genbank Accession Nos. XM_{—}346302 and XP_{—}346303.2, respectively; and, the predicted sequences of chimpanzee Bmx mRNA and protein are set out in Genbank Accession Nos. XM_{—}520948 and XP_{—}520948, respectively.

Bmx is found to be expressed predominantly in hematopoietic progenitor and myeloid hematopoietic cell lineages, and is also highly expressed in human heart endocardium and, importantly, in the endothelium of large arteries. Bmx is expressed in a subset of hematopoietic cells [Kaukonen et al., Br. J. Haematol. (1996) 94:455-460; Weil et al., Blood (1997) 90, 4332-4340]. In cultured mouse myeloid progenitor cells, Bmx regulated granulocyte-colony stimulating factor (G-CSF)-mediated granulocytic differentiation (Ekman et al., Circulation (2000) 96:1729-1732). In addition, Bmx expression was found in certain nonhematopoietic cells. As stated above, Bmx is highly expressed in cells with great migratory potential including certain metastatic tumor cells and endothelial cells (EC). [Chen et al., Nat Cell Biol (2001) 3:439-444; Ekman et al., 1997, supra; Bagheri-Yarmand et al., supra].

Bmx is activated by various angiogenic stimuli such as integrin engagement via focal adhesion kinase (FAK) (Chen et al., supra), VEGF via VEGFR-1, Tie-2 (Rajantie et al., supra), and VEGFR-2 [Chau et al, Oncogene (2002) 21:8817-29], and TNF via TNFR2 [Pan et al., Mol Cell Biol (2002) 22:7512-23] or/and via transactivation by VEGFR2 [Zhang et al., J Biol Chem (2003) 278:51267-76]. However, the mechanism by which Bmx mediates EC migration has not been determined. Several downstream effectors of Bmx involved in cell migration have been reported. Bmx, through its PH domain, directly binds to and activates Rho A by phpohsphorylating the protein (but not Rac1 and Cdc42) [Kim et al., J Biol Chem (2002) 277:30066-71]. Similarly, Bmx, through its PH domain, binds to and activates PAK1 [Bagheri-Yarmand et al., J Biol Chem. (2001) 276:29403-9], a 65-kDa serine/threonine kinase implicated in integrin-induced EC migration and angiogenesis by modulating EC contraction [Kiosses et al., Circ Res (2002) 90:697-702]. Bmx mediates the TNF-induced PI3K-Akt angiogenic pathway [Zhang et al., supra] which is involved in growth factor-stimulated cell migration. PI3K-Akt may induce angiogenesis by multiple downstream effectors including Rho family of small GTPase, PAK1 and endothelial nitric oxide synthase [Qi et al, supra; Gille et al., supra; Dimmeler et al., supra; Kureishi et al., supra; Matsumoto et al., Ackah et al., supra].

Based on data from Bmx activation by focal adhesion kinase (FAK), it has been proposed that integrin-induced binding of Bmx to FAK lead to phosphorylation of Bmx at Y40 to open up the “closed” conformation of the inactive Bmx and allow the kinase to be phosphorylated by Src family kinases at the highly conserved tyrosine residue Y566 in the catalytic domain [Qiu et al., Proc Natl Acad Sci USA (2002) 95:3644-49; Chen et al., supra]. Bmx activation has also been detected based on its translocation from the cytosol to the cellular membrane fraction [Zhang et al., Am J Physiol Heart Circ. Physiol. (2004) 287:H2364-66]. Bmx binds the membrane through its pleckstrin homology domain (Zhang et al., 2004, supra). Studies have indicated that stimulation of cardio-protective signals by administration of nitric oxide donors in rabbits increases the expression of Bmx tyrosine kinase protein and stimulates translocation and activation of Bmx tyrosine kinase (Zhang et al, 2004, supra).

A constitutively active form of Bmx (BMX-SK) (SEQ ID NO: 4) has been developed containing the SH2 and the kinase domains of the full-length Bmx protein. Other constitutively active variants of Bmx may be generated by deletion of the N-terminal pleckstrin homology domain, the Tec domain and/or the SH3 domain. Mutations in the kinase domain (K444Q in human Bmx) abolish kinase activity, and such variants are inactive. Additionally, mutation in the PH domain (E42K) removes membranes association of the protein and is inactivating, while deletion of the kinase domain renders the Bmx protein kinase inactive and non-functional.

Assays to measure Bmx activity of a variant Bmx protein include phosphotyrosine blots [Zhang et al., J Biol Chem (2003) 278:51267-76] and an immunocomplex assay to detect activity as described in Rajantie et al, (supra) and Ekman et al., (2001, supra)

As described herein in further detail, both genetic deficiency of Bmx mice (Bmx-KO) and endothelial-specific transgenic mice expressing a constitutively active of Bmx-SK (Bmx-SK-Tg) are used to examine the role of Bmx in inflammatory arteriogenesis and angiogenesis in an experimental ischemia model.

### Nucleic Acids of the Invention

The present invention provides isolated nucleic acid molecules (e.g., DNA or RNA) encoding Bmx tyrosine kinase and variants and fragments thereof that retain Bmx tyrosine kinase activity. In a preferred variation, the nucleic acids include suitable expression control and/or vector sequences for use in ex vivo or in vivo gene therapy.

The isolated polynucleotides of the invention include, but are not limited to, polynucleotides comprising the nucleotide sequences of SEQ ID NOs: 1 and 3, or a polynucleotide encoding any one of the peptide sequences of SEQ ID NOs: 2 or 4. A polynucleotide encoding a constitutively active form of Bmx tyrosine kinase is set out in SEQ ID NO: 3.

The polynucleotides useful in the present invention also include, but are not limited to, polynucleotides that encode polypeptides with BMX tyrosine kinase activity and that hybridize under stringent hybridization conditions to the complement of either (a) the nucleotide sequence of SEQ ID NOs: 1 or 3 or (b) a nucleotide sequence encoding the amino acid sequence of SEQ ID NOs: 2 or 4; a polynucleotide which is an allelic variant of any polynucleotide recited above; a polynucleotide which encodes a species homolog of any of the proteins recited above; or a polynucleotide that encodes a polypeptide comprising a specific domain or truncation of the BMX polypeptide of SEQ ID NO: 2. A polynucleotide encoding a constitutively active fragment of Bmx set out in SEQ ID NO: 4 is also contemplated.

The polynucleotides useful in the methods of the invention additionally include the complement of any of the polynucleotides recited above. The invention also encompasses allelic variants of the disclosed polynucleotides or proteins; that is, naturally-occurring alternative forms of the isolated polynucleotide which also encode proteins which are identical, homologous or related to that encoded by the polynucleotides.

In one embodiment, the nucleic acid molecule encoding a Bmx tyrosine kinase that is at least 70%, 75%, 80%, 85%, 90%, 95%, 96% 97%, 98% or 99% identical to a Bmx polynucleotide set out in SEQ ID NO: 1 or 3 is contemplated. Likewise, sequence variants of the constitutively active forms of Bmx inase are specifically contemplated. Nucleic acid molecules of the invention include nucleic acids that hybridize under highly stringent conditions, such as those described herein, to a Bmx nucleic acid sequence set out herein. Methods and algorithms for obtaining such polynucleotides are well known to those of skill in the art and can include, for example, methods for determining hybridization conditions which can routinely isolate polynucleotides of the desired sequence identities.

The nucleic acid sequences useful in the methods of the invention are further directed to sequences which encode variants of the described nucleic acids. These sequence variants may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence variants are preferably constructed by mutating the polynucleotide to encode an altered amino acid sequence. In one aspect, polynucleotides encoding the amino acid sequences are changed via site-directed mutagenesis, which is well known to those of skill in the art (see e.g., Edelman et al., DNA 2:183 (1983)). A further technique for generating amino acid variants is the cassette mutagenesis technique described in Wells et al., Gene 34:315 (1985); and other mutagenesis techniques well known in the art, such as, for example, the techniques in Sambrook et al., supra, and Current Protocols in Molecular Biology, Ausubel et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

Polynucleotides encoding preferred polypeptide truncations of the invention could be used to generate polynucleotides encoding chimeric or fusion proteins comprising one or more domains of the polynucleotides or polypeptides useful in the methods of the invention and heterologous protein sequences.

A polynucleotide according to the invention can be joined to any of a variety of other nucleotide sequences by well-established recombinant DNA techniques (see Sambrook J et al. (1989) Molecular Cloning: A Laboratory Manual, Cold Spring Harbor Laboratory, NY). Useful nucleotide sequences for joining to polypeptides include an assortment of expression control sequences and vectors, e.g., plasmids, cosmids, lambda phage derivatives, phagemids, and the like, many of which are now known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having the sequence set out in SEQ ID NOs: 1 or 3 or fragments thereof or any other polynucleotides useful in the methods of the invention. In one embodiment, the recombinant constructs of the present invention comprise a vector, such as a plasmid or viral vector, into which a nucleic acid having the sequence of any one of SEQ ID NOs: 1 or 3 or a fragment thereof is inserted, in a forward or reverse orientation. Large numbers of suitable vectors and promoters are known to those of skill in the art and are commercially available for generating the recombinant constructs of BMX tyrosine kinase. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, pSVL (Pharmacia).

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990).

### Polypeptides of the Invention

Polynucleotides as described above can be expressed in vitro to confirm that Bmx tyrosine kinase activity is retained for the encoded polypeptides. Such procedures are particularly useful to identify variants or naturally occurring wild type sequences and previously characterized constiutively active Bmx fragments. The isolated polypeptides useful in the methods of the invention include, but are not limited to, a polypeptide comprising the amino acid sequence of SEQ ID NOs: 2 or 4 or the amino acid sequence encoded by the DNA of SEQ ID NOs: 1 or 3 or a portion thereof corresponding to biologically active fragment of Bmx. Polypeptides useful in the invention also include polypeptides with BMX tyrosine kinase activity that are encoded by any of the polynucleotides described herein. Biologically active variants of the BMX tyrosine kinase protein sequences of SEQ ID NOs: 2 or 4 (e.g., at least 70%, 75%, 80%, 85%, 90%, 95%, 96% 97%, 98% or 99% amino acid identity) that retain BMX tyrosine kinase activity are contemplated.

With respect to the Bmx tyrosine kinase used to practice the invention, native sequences will usually be most preferred. By “native sequences” is meant sequences encoded by naturally occurring polynucleotides, including but not limited to prepro-peptides, pro-peptides, and partially and fully proteolytically processed polypeptides. Naturally occurring sequences from the same species as the organism that is to be treated is highly preferred. For purposes described herein, fragments of the forgoing that retain the tyrosine kinase activities of interest also shall be considered native sequences. Moreover, modifications can be made to most protein sequences without destroying the activity of interest of the protein, especially conservative amino acid substitutions, and proteins so modified are also suitable for practice of the invention. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

Moreover, deletion and addition of amino acids is often possible without destroying a desired activity. With respect to the present invention, where activity is of particular interest and the ability of Bmx tyrosine kinase expressed through gene therapy to induce angiogenesis or arteriogenesis is of special interest, cell based assays and tyrosine phosphorylation assays are available to determine whether a particular Bmx variant (a) has tyrosine kinase activity and (b) stimulates or inhibits downstream signaling that leads to angiogenic or arteriogenic processes.

Fragments of the proteins of the present invention which are capable of exhibiting biological activity are also encompassed by the present invention. The relevant domains of the Bmx protein have been determined and are available on the Swiss-Prot database, maintained by a collaboration between the Swiss Institute of Bioinformatics and the EMBL outstation—the European Bioinformatics Institute. For example, the pleckstrin homology domain is found from approximately residues 4-111 of SEQ ID NO: 2, while the SH2 domain is located at approximately residues 296-392 of SEQ ID NO: 2 and the Bmx protein kinase domain is located at approximately residues 417 to 675 of SEQ ID NO: 2. The Bmx-SK construct contemplated herein comprises the SH2 domain and the kinase domain and is set out in SEQ ID NO: 4.

Two manners for defining genera of polypeptide variants include percent amino acid identity to a native polypeptide (e.g., 80, 85, 90, 91, 92, 93, 94, 95, 96, 97, 98, or 99% identity preferred, as determined by methods standard in the art such as BLAST, which stands for Basic Local Alignment Search Tool, is used to search for local sequence alignments (Altschul SF (1993) J Mol Evol 36:290-300; Altschul, SF et al (1990) J Mol Biol 215:403-10).), or the ability of encoding-polynucleotides to hybridize to each other under specified conditions. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.). Other exemplary hybridization conditions are described herein in the examples. One exemplary set of conditions is as follows: hybridization at 42° C. in 50% formamide, 5× SSC, 20 mM Na.PO4, pH 6.8; and washing in 1× SSC at 55° C. for 30 minutes. Formula for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press Cold Spring Harbor, N.Y. (1989), pp. 9.47 to 9.51.

The invention also contemplates methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

In an alternative method, the polypeptide or protein is purified from bacterial cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size exclusion chromatography, ion exchange chromatography, and immuno affinity chromatography. See, e.g., Scopes, Protein Purification: Principles and Practice, Springer Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

The protein may also be produced by operably linking the isolated polynucleotide of the invention to suitable control sequences in one or more insect expression vectors, and employing an insect expression system. Materials and Methods for baculovirus/insect cell expression systems are commercially available in kit form from, e.g., Invitrogen, San Diego, Calif., U.S.A. (the MAXBAT™ kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

Alternatively, the protein of the invention may also be expressed in a form which will facilitate purification. For example, it may be expressed as a fusion protein, such as those of maltose binding protein (MBP), glutathione S transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“FLAG®”) is commercially available from Kodak (New Haven, Conn.).

The invention also provides chimeric or fusion proteins. As used herein, a “chimeric protein” or “fusion protein” comprises a polypeptide of the invention operatively linked to another polypeptide. Within a fusion protein the polypeptide according to the invention can correspond to all or a portion of a protein according to the invention. In one embodiment, a fusion protein comprises at least one biologically active portion of a protein according to the invention. In another embodiment, a fusion protein comprises at least two biologically active portions of a protein according to the invention. Within the fusion protein, the term “operatively linked” is intended to indicate that the polypeptide according to the invention and the other polypeptide are fused in frame to each other. The polypeptide can be fused to the N terminus or C terminus, or to the middle.

For example, in one embodiment a fusion protein comprises a polypeptide according to the invention operably linked to the extracellular domain of a second protein.

A chimeric or fusion protein of the invention can be produced by standard recombinant DNA techniques. For example, DNA fragments coding for the different polypeptide sequences are ligated together in frame in accordance with conventional techniques, e.g., by employing blunt ended or stagger ended termini for ligation, restriction enzyme digestion to provide for appropriate termini, filling in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols In Molecular Biology, John Wiley & Sons, 1992). Moreover, many expression vectors are commercially available that already encode a fusion moiety (e.g., a GST polypeptide). A nucleic acid encoding a polypeptide of the invention can be cloned into such an expression vector such that the fusion moiety is linked in frame to the protein of the invention.

### Vascular Disease

Materials and methods of the invention are useful for prophylaxis or therapy for a variety of diseases associated with poor circulation including those described in this section.

Peripheral arterial disease (also called PAD, peripheral vascular disease, peripheral arterial occlusive disease (PAOD) or arteriosclerosis) is a problem with blood flow in the arteries. Arteriosclerosis of the extremities is a disease of the blood vessels characterized by narrowing and hardening of the arteries that supply the legs and feet. This causes a decrease in blood flow that can injure nerves and other tissues. The most common cause of narrow or blocked arteries is the buildup of fatty deposits, or atherosclerosis. The most common complaint of people who have PAD or arteriosclerosis is claudication. Claudication is pain in the calf or thigh muscle that occurs during walking. Claudication occurs when the artery supplying blood to the extremities narrows due to PAD and not enough blood is flowing to a muscle.

A Doppler study is used to check blood flow in the legs. With this test, blood pressure cuffs are wrapped around an arm and a leg on the same side of the body. Four cuffs are wrapped around the leg—one at the upper thigh, one at the lower thigh, one at the upper calf and one at the ankle—to measure the blood pressure from the top of the leg to the ankle. A cuff is also wrapped around the upper arm to measure the blood pressure. The blood pressure in the arm is compared with the blood pressure in the leg, and a drop in leg blood pressure may indicate narrowing of an artery. Agents that are used to treat PAD and claudication include pentoxifylline or cilostazol. Other medications may be required to control the disorder, including pain relievers, blood thinners, and medications to enlarge or dilate the affected artery(ies).

Coronary artery disease (CAD) occurs when the arteries that supply blood to the heart muscle (the coronary arteries) become hardened and narrowed. The arteries harden and narrow due to buildup of plaque on their inner walls, known as atherosclerosis. As the plaque increases in size, the insides of the coronary arteries get narrower, less blood can flow through, blood flow to the heart muscle is reduced, and, because blood carries much-needed oxygen, the heart muscle is not able to receive the amount of oxygen it needs. Reduced or cutoff blood flow and oxygen supply to the heart muscle can result in angina or heart attack.

Initial screening for CAD commonly involves stressing the heart under controlled conditions to detect the presence of flow-limiting blockages in the coronary arteries, generally in the range of at least a 50% reduction in the diameter of at least one of the three major coronary arteries. There are two basic types of stress tests; those that involve exercising the patient to stress the heart (exercise cardiac stress tests), and those that involve chemically stimulating the heart directly to mimic the stress of exercise (physiologic stress testing). In an exercise stress test, the patient's electrocardiogram (EKG), heart rate, heart rhythm, and blood pressure are continuously monitored. If a coronary arterial blockage results in decreased blood flow to a part of the heart during exercise, certain changes may be observed in the EKG, as well as in the response of the heart rate and blood pressure.

Radionucleide stress testing involves injecting a radioactive isotope (typically thallium or cardiolyte) into the patient's vein after which an image of the patient's heart becomes visible. The radioactive isotopes are absorbed by the normal heart muscle. Nuclear images obtained in the resting condition, are compared with those taken immediately following exercise. During exercise, if a blockage in a coronary artery results in diminished blood flow to a part of the cardiac muscle, this region of the heart will appear as a relative “cold spot” on the nuclear scan.

During a physiologic stress test, medications are administered which stimulate the heart to mimic the physiologic effects of exercise. For example, dobutamine, which is similar to adrenaline, is carefully administered to gradually increase the heart rate and strength of the contractions of the heart muscle. Simultaneously, echocardiography or radionucleide imaging is performed. Alternatively, adenosine is administered, which simulates the physiology of the coronary artery circulation during exercise.

Several types of medicine are commonly used to treat CAD, including: cholesterol-lowering medicines, anticoagulants, aspirin, and other antiplatelet medicines, which help to prevent clots from forming in arteries and blocking blood flow; ACE (angiotensin-converting enzyme) inhibitors which help to lower blood pressure; Beta blockers, which slow heart rate and lower blood pressure to decrease the workload on the heart; Calcium channel blockers, which relax blood vessels and lower blood pressure; Nitroglycerin, which dilates the coronary arteries, increasing blood flow to the heart; long-acting nitrates; Glycoprotein IIb-IIIa inhibitors, which are very strong antiplatelet medicines used during and after angioplasty or to treat angina; thrombolytic agents, which dissolve clots that can occur during a heart attack.

Arterial stenosis is narrowing or blockage of any one of the major arteries in the body, such as the renal arterial stenosis in the artery that supplies the kidney, and carotid artery stenosis in the arteries that supply the brain. Arterial stenosis is typically caused by atherosclerosis.

Cerebrovascular disease (CVD) includes all disorders in which an area of the brain is transiently or permanently affected by ischemia (restricted blood flow and oxygen to a part of the body) or bleeding and one or more of the cerebral blood vessels are involved in the pathological process. Cerebrovascular disease includes stroke, carotid stenosis, vertebral stenosis and intracranial stenosis, aneurysms, and vascular malformations.

The major arteries involved in cerebrovascular disease include the carotid arteries, the basilar artery, the cerebral arteries and the vertebral arteries. Atherosclerosis of any of these arteries can interrupt intracranial or extracranial arterial blood flow and impair collateral flow, causing brain ischemia and consequent neurologic symptoms. If the blood supply is promptly restored, brain tissues recover and symptoms disappear, but if ischemia lasts longer than 1 h, infarction and permanent neurologic damage result. Thrombi or emboli due to atherosclerosis or other disorders (eg, arteritis, rheumatic heart disease) commonly cause ischemic arterial obstruction.

Hypertension, atherosclerosis, heart disease, atrial fibrillation, diabetes mellitus, and polycythemia predispose an individual to transient ischemic attacks in the brain. Treatment of transient ischemic attacks includes antiplatelet drugs or anticoagulants when the obstruction is intracranial or vertebrobasilar or when both vertebral and carotid arteries are affected, provided the patient is not hypertensive. Heparin is used initially for recent daily attacks; a warfarin derivative can be used for less frequent attacks. The duration of anticoagulant therapy is empiric; often, anticoagulants are continued for 2 to 3 months before a trial without therapy. Aspirin or ticlopidine is also used.

Ischemia is a condition in which the blood flow (and thus oxygen) is restricted to a part of the body. Cardiac ischemia is the name for lack of blood flow and oxygen to the heart muscle.

In ischemic stroke, the middle cerebral artery or one of its deep penetrating branches is most commonly occluded. Recombinant tissue plasminogen activator (tPA), given within 3 h of symptom onset, can improve neurologic outcome of selected acute stroke patients. Anticoagulation drugs are typically administered to prevent subsequent strokes.

Several animal models are available to monitor ischemic injury that represent injury and arterial disease in human patients. Briefly, in ischemic injury models the proximal end of the left femoral artery and the distal portion of saphenous artery are ligated. All branches between these two sites are ligated or cauterized, and arteriectomy is performed. Studies using this mouse model, consistent with data from patients with PAD, reveal that a reduction in calf blood pressure ratios and blood flow correlate with a marked decrease in the capillaries surrounding each muscle fiber (capillary/fiber ratio) indicative of the inability to mount an adaptive angiogenic response. For example, Tanii et al (Circ Res. 2006 98:55-62) have described measuring ischemic disease as a result of diabetes mellitus induced in experimental animals. Also, other studies using ischemic mouse models have been used to assess levels of angiogeneic factors as a result of varied treatments. See e.g., Liddell et al., (J Vasc Interv Radiol. 2005 7:991-8), which describes administration of G-CSF to ischemic rabbits, Gounis et al., (Gene Ther. 2005 12:762-71), which describes moderate effects of VEGF gene therapy in ischemic rabbits, and Stabile et al. (Circulation. 2003 108:205-10), which assessed the effects of lymphocyte cells on angiogenesis in knockout mice.

Thus the ischemia hindlimb model is a very useful demonstrate the efficacy of gene therapy for inflammatory arteriogenesis/angiogenesis.

### Gene Therapy

The therapeutic effects of the Bmx gene on arteriogenesis and angiogenesis are achieved by administration of Bmx-encoding polynucleotides (including vectors comprising such polynucleotides) to a subject that will benefit from administration of the Bmx gene.

A gene therapy construct can comprise any suitable vector, including but not limited to viruses, plasmids, water-oil emulsions, polyethylene imines, dendrimers, micelles, microcapsules, liposomes, and cationic lipids. Where appropriate, two or more types of vectors can be used together. For example, a plasmid vector can be used in conjunction with liposomes. See e.g., U.S. Pat. No. 5,928,944.

For these embodiments, an exemplary expression construct comprises a virus or engineered construct derived from a viral genome. The expression construct generally comprises a nucleic acid encoding the gene to be expressed and also additional regulatory regions that will effect the expression of the gene in the cell to which it is administered. Such regulatory regions include for example promoters, enhancers, introns, polyadenylation signals, 3′ UTR sequences, and the like. Also contemplated in regulation of Bmx expression are methods to stabilize the protein product such as chemical inhibitors of Bmx degradation (e.g., proteosome inhibitors), or mutations of the Bmx sequence and also methods that specifically turn on stabilizing the Bmx gene, such as zinc-finger constructs known in the art.

DNA may be introduced into a cell using a variety of viral vectors. In such embodiments, expression constructs comprising viral vectors containing the genes of interest may be adenoviral (see, for example, U.S. Pat. No. 6,908,762, U.S. Pat. No. 6,756,226, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see, for example, U.S. Pat. No. 6,821,776, U.S. Pat. No. 6,808,922, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), lentiviral (see, for example, U.S. Pat. No. 6,800,281, U.S. Pat. No. 6,277,633), adeno-associated viral (see, for example, U.S. Pat. No. 6,489,162, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see, for example, U.S. Pat. No. 6,387,368, U.S. Pat. No. 5,856,152 and Goncalves et al., (Virology. 2001, 288:236-46, incorporated herein by reference) or a vaccinia viral or a herpesviral (see, for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector. Vectors have also been described which involve encapsulating genes or proteins in liposomes and fusing this with inactivated HVJ (hemagglutinating virus such as Sendai virus) to create fusion particles, as in conventional HVJ-liposome, have enabled a non-invasive gene transfer into cultured cells or in vivo tissue. This technique is in frequent use in animal models (Dzau et al., Proc. Natl. Acad. Sci. USA (1996) 93:11421-25; Kaneda et al., Molecular Medicine Today (1999) 5:298-303). A vector comprising a virus envelope vector (HVJ virus) which is capable of gene transfer has been described in US Patent Publication No. 20050239188. for each category of vector, replication-deficient forms represent a preferred variation.

A gene therapy construct of the present invention can also employ an inducible promoter. For example, a tetracycline responsive promoter has been used effectively to regulate transgene expression in rat brain (Mitchell & Habermann, 1999 Biol Res Nurs 1:12-19). Other inducible promoters include hormone-inducible promoters (No et al., Proc Natl Acad Sci USA (1996) 93:3346-51.; Abruzzese et al., Hum Gene Ther (1999) 10:1499-1507.; Burcin et al., Proc Natl Acad Sci USA (1999) 96:355-360), radiation-inducible promoters, such as those employing the Egr-1 promoter or NF-.quadrature.B promoter (Weichselbaum et al., J Natl Cancer Inst (1991) 83:480-484.; Weichselbaum et al., Int J Radiat Oncol Biol Phys (1994) 30:229-234), and heat-inducible promoters (Madio et al., J Magn Reson Imaging (1998) 8:101-104.; Gerner et al., Int J Hyperthermia (2000) 16:171-181.; Vekris et al., J Gene Med (2000) 2:89-96). Promoters contemplated by the invention include, but are not limited to, ubiquitous promoters such as the cytomegalovirus (CMV) promoter/enhancer, long terminal repeat (LTR) of retroviruses, the γ-actin promoter and the like, tissue specific promoters such as Tie-2, VE-cadherin and other endothelial cell and bone marrow-specific promoters, and inducible promoters such as the tetracycline (Tet) promoter.

Other expression control sequences contemplated for use in the invention include enhancers, introns, polyadenylation signal, and 3′UTR sequences.

In other embodiments, non-viral delivery is contemplated. These include calcium phosphate precipitation (Graham and Van Der Eb, Virology, 52:456-467 (1973); Chen and Okayama, Mol. Cell Biol., 7:2745-2752, (1987); Rippe, et al., Mol. Cell Biol., 10:689-695 (1990)), DEAE-dextran (Gopal, Mol. Cell Biol., 5:1188-1190 (1985)), electroporation (Tur-Kaspa, et al., Mol. Cell Biol., 6:716-718, (1986); Potter, et al., Proc. Nat. Acad. Sci. USA, 81:7161-7165, (1984)), direct microinjection (Harland and Weintraub, J Cell Biol., 101:1094-1099 (1985)), DNA-loaded liposomes (Nicolau and Sene, Biochim. Biophys. Acta, 721:185-190 (1982); Fraley, et al., Proc. Natl. Acad. Sci. USA, 76:3348-3352 (1979); Feigner, Sci. Am., 276(6):102-6 (1997); Feigner, Hum. Gene Ther., 7(15):1791-3, (1996)), cell sonication (Fechheimer, et al., Proc. Natl. Acad. Sci. USA, 84:8463-8467 (1987)), gene bombardment using high velocity microprojectiles (Yang, et al., Proc. Natl. Acad. Sci. USA, 87:9568-9572 (1990)), and receptor-mediated transfection (Wu and Wu, J. Biol. Chem., 262:4429-4432 (1987); Wu and Wu, Biochemistry, 27:887-892 (1988); Wu and Wu, Adv. Drug Delivery Rev., 12:159-167 (1993)).

In a particular embodiment of the invention, the expression construct (or indeed the peptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, “In Liver Diseases, Targeted Diagnosis And Therapy Using Specific Receptors And Ligands,” Wu, G., Wu, C., ed., New York: Marcel Dekker, pp. 87-104 (1991)). The addition of DNA to cationic liposomes causes a topological transition from liposomes to optically birefringent liquid-crystalline condensed globules (Radler, et al., Science, 275(5301):810-4, (1997)). These DNA-lipid complexes are potential non-viral vectors for use in gene therapy and delivery.

Liposome-mediated nucleic acid delivery and expression of foreign DNA in vitro has been very successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda, et al., Science, 243:375-378 (1989)). In other embodiments, the liposome may be complexed or employed in conjunction with nuclear nonhistone chromosomal proteins (HMG-1) (Kato, et al., J. Biol. Chem., 266:3361-3364 (1991)). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky, et al., Proc. Nat. Acad. Sci. USA, 81:7529-7533 (1984) successfully injected polyomavirus DNA in the form of CaPO₄ precipitates into liver and spleen of adult and newborn mice demonstrating active viral replication and acute infection. Benvenisty and Neshif, Proc. Nat Acad. Sci. USA, 83:9551-9555 (1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein, et al., Nature, 327:70-73 (1987)). Several devices for accelerating small particles have been developed. One such device relies on a high voltage discharge to generate an electrical current, which in turn provides the motive force (Yang, et al., Proc. Natl. Acad. Sci USA, 87:9568-9572 (1990)). The microprojectiles used have consisted of biologically inert substances such as tungsten or gold beads.

Genetic control can also be achieved through the design of novel transcription factors for modulating expression of the gene of interest in native cells and animals. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular target sequence of the present invention facilitates the engineering of zinc finger proteins specific for the target sequence using known methods such as a combination of structure-based modeling and screening of phage display libraries [Segal et al., (1999) Proc Natl Acad Sci USA 96:2758-2763; Liu et al., (1997) Proc Natl Acad Sci USA 94:5525-30; Greisman and Pabo (1997) Science 275:657-61; Choo et al., (1997) J Mol Biol 273:525-32]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., (1999) Proc Natl Acad Sci USA 96:2758-2763]. The artificial zinc finger repeats, designed based on target sequences, are fused to activation or repression domains to promote or suppress gene expression [Liu et al., (1997) Proc Natl Acad Sci USA 94:5525-30]. Alternatively, the zinc finger domains can be fused to the TATA box-binding factor (TBP) with varying lengths of linker region between the zinc finger peptide and the TBP to create either transcriptional activators or repressors [Kim et al., (1997) Proc Natl Acad Sci USA 94:3616-3620]. Such proteins, and polynucleotides that encode them, have utility for modulating expression in vivo in both native cells, animals and humans. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., (1999) Proc Natl Acad Sci USA 96:9521-6; Wu et al., (1995) Proc Natl Acad Sci USA 92:344-348].

Various routes of administration are contemplated for various cell types. For practically any cell, tissue or organ type, systemic delivery is contemplated. In other embodiments, a variety of direct, local and regional approaches may be taken. For example, the cell, tissue or organ may be directly injected with the expression vector or protein.

In a different embodiment, ex vivo gene therapy is contemplated. In an ex vivo embodiment, cells from the patient are removed and maintained outside the body for at least some period of time. During this period, a therapy is delivered, after which the cells are reintroduced into the patient; preferably, any tumor cells in the sample have been killed. In the present invention, it is contemplated that endothelial cells, endothelial precursor cells, and stem cells that may differentiate into endothelial cells are transfected with a Bmx gene vector. Also contemplated are bone-marrow derived cells, including monocytes and macrophages.

### Pharmaceutical Formulations

In the case where the gene transfer vector according to the present invention is employed as a composition for gene therapy, the administration of the vector according to the present invention may be achieved through direct injection of a vector suspension which is suspended in PBS (phosphate buffered saline), saline, etc., to local sites (e.g. intra-cancerous tissue, intrahepatic, intramuscular and intracerebral), or through intravascular administration (e.g., intraarterial, intravenous or intraportal) thereof.

In one embodiment, the gene transfer vector may be formulated generally by mixing the gene transfer vector, in a unit dosage injectable form (solution, suspension or emulsion), with a pharmaceutically acceptable carrier (i.e., one that is non-toxic to recipients at the dosages and concentrations employed and is compatible with other ingredients of the formulation). For example, the formulation preferably does not include oxidizing agents and other compounds that are known to be deleterious to the gene transfer vector.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins (such as serum albumin, gelatin, or immunoglobulins); hydrophilic polymers (such as polyvinylpyrrolidone); amino acids (such as glycine, glutamic acid, aspartic acid, or arginine; monosaccharides, disaccharides, and other carbohydrates (including cellulose or its derivatives, glucose, mannose, or dextrins); chelating agents (such as EDTA); sugar alcohols (such as mannitol or sorbitol); counterions (such as sodium); and/or nonionic detergents (such as polysorbates or poloxamers), or PEG.

A pharmaceutical composition containing a gene transfer vector may typically be stored as an aqueous solution in a unit- or multi-dosage container, e.g., sealed ampule or vial.

In some embodiments, the gene therapy vector is delivered locally, e.g., via balloon catheter or an a surface of a drug-eluting stent or membrane coated stent. Various, polymers, coatings, suspensions, and formulations for drug delivering stents and the like are contemplated for delivering a gene therapy composition of the invention. See e.g., Klugherz et al., Hum Gene Ther (2002) 13:443-54; Perlstein at al., Gene Ther (2003) 10:1420-1428; and Takahashi et al, Gene Ther (2003) 10:1471-78 (all of which are incorporated herein by reference).

The present invention also provides a pharmaceutical package or kit including one or more containers filled with one or more ingredients of the pharmaceutical composition according to the present invention. Furthermore, the polypeptide according to the present invention may be used along with other therapeutic compounds.

### Dosing

A pharmaceutical composition containing the Bmx gene transfer vector according to the present invention is formulated and dosed in a fashion consistent with good medical practice, taking into account the clinical condition of the individual patient (e.g., condition to be prevented or treated), the site of delivery of the composition containing the gene transfer vector, the target tissue, the administration method, the scheduling of administration, and other factors known to those skilled in the art. Accordingly, an “effective amount” or an appropriate dosage of the gene transfer vector described in the present specification is determined by such considerations.

Dose and dosing also can be modulated with monitoring of the patient. Monitoring of a patient receiving treatment is performed by the treating physician. Symptoms or factors monitored in patients receiving Bmx therapy to determine if there has been an increase in arteriogeneis and/or angiogenesis include measurement of local blood flow at the site of arterial injury, vascular resistance, tissue recovery and exercise capability. Monitoring tests are similar to those tests used to diagnose arterial disease, such as Doppler screens, and others described above. Moreover, dose and dosing are modulated depending on whether permanent or transient transfection is desired, and on the duration of transgene expression in transiently transfected cells.

For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

The “administering” that is performed according to the present invention may be performed using any medically-accepted means for introducing a therapeutic directly or indirectly into a mammalian subject, including but not limited to injections (e.g., intravenous, intramuscular, subcutaneous, or catheter). For some cardiovascular diseases a preferred route of administration is intravascular, such as by intravenous, intra-arterial, or intracoronary arterial injection. In one embodiment, administering the composition is performed at the site of a lesion or affected tissue needing treatment by direct injection into the lesion site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the lesion.

The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

Dosing may be modified if traditional therapeutics are administered in combination with therapeutics of the invention. For example, treatment of arterial disease using traditional agents known in the art, or a second agent as referred to herein, in combination with methods of the invention, is contemplated. Second agents contemplated by the invention include agents commonly used in the art to treat vascular diseases and those described herein.

Examples of agents contemplated as second agents include, but are not limited to, agents used to treat PAD such as pentoxifylline or cilostazol, pain relievers, blood thinners, medications to enlarge or dilate the affected artery. Also contemplated are agents used to treat CAD, including: cholesterol-lowering medicines, anticoagulants, aspirin, and other antiplatelet medicines, ACE (angiotensin-converting enzyme), Beta blockers, Calcium channel blockers, Nitroglycerin, long-acting nitrates, Glycoprotein IIb-IIIa inhibitors, and thrombolytic agents. Also contemplated are warfarin or ticlopidine.

In a further aspect, it is contemplated that the Bmx gene therapy vector may be administered with cell-permeable peptides, such as the third alpha-helix of the Antennapedia (Antp) homeodomain or a fragment of HIV Tat protein, which provide improved transmission across the cell membrane through a receptor-independent mechanism. [Schwarze et al., Science (1999) 285:1569-1572; Derossi et al., Trends Cell Biol. (1998) 8:84-87.

It is contemplated that the Bmx gene therapy vector and the second agent may be given concurrently, with concurrently referring to agents given within 30 minutes of each other. In another aspect, the second agent is administered prior to administration of Bmx gene therapy vector composition. Prior administration refers to administration of the second agent within the range of six weeks prior to treatment with the Bmx gene therapy vector, up to 30 minutes before administration of the vector. It is further contemplated that the second agent is administered subsequent to administration of the Bmx gene therapy vector composition. Subsequent administration is meant to describe administration from 30 minutes after Bmx gene therapy vector treatment up to two, three or four weeks after administration of gene therapy vector.

### Methods for Administration

It is contemplated that the gene therapy vector is administered to a subject locally or systemically. In one aspect, administering comprises a catheter-mediated transfer of the transgene-containing composition into a blood vessel of the mammalian subject, especially into a coronary artery of the mammalian subject. Exemplary materials and methods for local delivery are reviewed in Lincoff et al., Circulation, 90: 2070-2084 (1994); and Wilensky et al., Trends Cardiovasc. Med., 3:163-170 (1993), both incorporated herein by reference. For example, the composition is administered using infusion-perfusion balloon catheters (preferably mircroporous balloon catheters) such as those that have been described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; and Wolinsky et al., J. Am. Coll. Cardiol., 15: 475-481 (1990) (Wolinsky Infusion Catheter); and Lambert et al., Coron. Artery Dis., 4: 469-475 (1993), all of which are incorporated herein by reference in their entirety. Use of such catheters for site-directed somatic cell gene therapy is described, e.g., in Mazur et al., Texas Heart Institute Journal, 21; 104-111 (1994), incorporated herein by reference. In an embodiment where the Bmx transgene is administered in an adenovirus vector, the vector is preferably administered in a pharmaceutically acceptable carrier at a titer of 10⁷-10¹³ viral particles, and more preferably at a titer of 10⁹-10¹¹ viral particles. The adenoviral vector composition preferably is infused over a period of 15 seconds to 30 minutes, more preferably 1 to 10 minutes.

For example, in patients with angina pectoris due to a single or multiple lesions in coronary arteries and for whom percutaneous transluminal coronary angioplasty (PTCA) is prescribed on the basis of primary coronary angiogram findings, an exemplary protocol involves performing PTCA through a 7 F guiding catheter according to standard clinical practice using the femoral approach. If an optimal result is not achieved with PTCA alone, then an endovascular stent also is implanted. (A nonoptimal result is defined as residual stenosis of >30% of the luminal diameter according to a visual estimate, and B or C type dissection.) Arterial gene transfer at the site of balloon dilatation is performed with a replication-deficient adenoviral Bmx vector immediately after the angioplasty, but before stent implantation, using an infusion-perfusion balloon catheter. The size of the catheter will be selected to match the diameter of the artery as measured from the angiogram, varying, e.g., from 3.0 to 3.5 F in diameter. The balloon is inflated to the optimal pressure and gene transfer is performed during a 10 minute infusion at the rate of 0.5 ml/min with virus titer of 1.15×10¹⁰.

In another embodiment, intravascular administration with a gel-coated catheter is contemplated, as has been described in the literature to introduce other transgenes. See, e.g., U.S. Pat. No. 5,674,192 (Catheter coated with tenaciously-adhered swellable hydrogel polymer); Riessen et al., Human Gene Therapy, 4: 749-758 (1993); and Steg et al., Circulation, 96: 408-411 (1997) and 90: 1648-1656 (1994); all incorporated herein by reference. Briefly, DNA in solution (e.g., the Bmx kinase polynucleotide) is applied one or more times ex vivo to the surface of an inflated angioplasty catheter balloon coated with a hydrogel polymer (e.g., Slider with Hydroplus, Mansfield Boston Scientific Corp., Watertown, Mass.). The Hydroplus coating is a hydrophilic polyacrylic acid polymer that is cross-linked to the balloon to form a high molecular weight hydrogel tightly adhered to the balloon. The DNA covered hydrogel is permitted to dry before deflating the balloon. Re-inflation of the balloon intravascularly, during an angioplasty procedure, causes the transfer of the DNA to the vessel wall.

In another preferred embodiment, the administering comprises implanting an intravascular stent in the mammalian subject, where the stent is coated or impregnated with the therapeutic Bmx gene composition. Exemplary materials for constructing a drug-coated or drug-impregnated stent are described in the art and reviewed in Lincoff et al., Circulation, 90: 2070-2084 (1994). As described in U.S. Pat. No. 6,958,147, a metal or polymeric wire for forming a stent is coated with a composition such as a porous biocompatible polymer or gel that is impregnated with (or can be dipped in or otherwise easily coated immediately prior to use with) a Bmx kinase therapeutic composition. The wire is coiled, woven, or otherwise formed into a stent suitable for implanation into the lumen of a vessel using conventional materials and techniques, such as intravascular angioplasty catheterization. Exemplary stents that may be improved in this manner are described and depicted in U.S. Pat. Nos. 5,800,507 and 5,697,967 (Medtronic, Inc., describing an intraluminal stent comprising fibrin and an elutable drug capable of providing a treatment of restenosis); U.S. Pat. No. 5,776,184 (Medtronic, Inc., describing a stent with a porous coating comprising a polymer and a therapeutic substance in a solid or solid/solution with the polymer); U.S. Pat. No. 5,799,384 (Medtronic, Inc., describing a flexible, cylindrical, metal stent having a biocompatible polymeric surface to contact a body lumen); U.S. Pat. Nos. 5,824,048 and 5,679,400; and U.S. Pat. No. 5,779,729; all of which are specifically incorporated herein by reference in the entirety. Implantation of such stents during conventional angioplasty techniques will result in less restenosis than implantation of conventional stents. In this sense, the biocompatibility of the stent is improved.

In another embodiment, the composition comprises microparticles composed of biodegradable polymers such as PGLA, non-degradable polymers, or biological polymers (e.g., starch) which particles encapsulate or are impregnated by a composition comprising the Bmx transgene. Such particles are delivered to the intravascular wall using, e.g., an infusion angioplasty catheter. Other techniques for achieving locally sustained drug delivery are reviewed in Wilensky et al., Trends Cardiovasc. Med., 3:163-170 (1993), incorporated herein by reference.

In yet another embodiment, an expandable elastic membrane or similar structure mounted to or integral with a balloon angioplasty catheter or stent is employed to deliver the Bmx transgene. See, e.g., U.S. Pat. Nos. 5,707,385, 5,697,967, 5,700,286, 5,800,507, and 5,776,184, all incorporated by reference herein. As described in PCT/EP2004/012406, a single layer or multi-layer sheet of elastic membrane material is formed into a tubular structure, e.g., by bringing together and adhering opposite edges of the sheet(s), e.g., in an overlapping or a abutting relationship. In this manner the elastomeric material may be wrapped around a catheter balloon or stent. A therapeutic BMX transgene composition is combined with the membrane using any suitable means, including injection molding, coating, diffusion, and absorption techniques. In the multilayer embodiment depicted in the figures, the edges of the two layers may be joined to form a fluid-tight seal. In some embodiments, one layer of material is first processed by stretching the material and introducing a plurality of microscopic holes or slits. After the layers have been joined together, the sheet can be stretched and injected with the therapeutic composition through one of the holes or slits to fill the cavity that exists between the layers. The sheet is then relaxed, causing the holes to close and sealing the therapeutic composition between the layers until such time as the sheet is again stretched. This occurs, for example, at the time that an endovascular stent or balloon covered by the sheet is expanded within the lumen of a stenosed blood vessel. The expanding stent or balloon presses radially outward against the inner surface of the tubular sheet covering, thus stretching the sheet, opening the holes, and delivering the therapeutic agent to the walls of the vessel.

In another variation, the composition containing the Bmx transgene is administered extravascularly, e.g., using a device to surround or encapsulate a portion of vessel. See, e.g., International Patent Publication WO 98/20027, incorporated herein by reference, describing a collar that is placed around the outside of an artery (e.g., during a bypass procedure) to deliver a transgene to the arterial wall via a plasmid or liposome vector. As further described in PCT/EP2004/012406, an extravascular collar including a void space is defined by a wall formed, e.g., of a biodegradable or biocompatible material. The collar touches the wall of a blood vessel at the collar's outer extremities. Blood flows through the lumen of the blood vessel. A longitudinal slit in the flexible collar permits the collar to be deformed and placed around the vessel and then sealed using a conventional tissue glue, such as a thrombin glue.

In still another variation, endothelial cells or endothelial progenitor cells are transfected ex vivo with the Bmx transgene, and the transfected cells as administered to the mammalian subject. Exemplary procedures for seeding a vascular graft with genetically modified endothelial cells are described in U.S. Pat. No. 5,785,965, incorporated herein by reference.

### Medical Devices

An additional aspect of the invention is a medical device, such as any of the catheters, stents, membranes, collars, syringes, or other devices for delivering that contains, is coated with, impregnated with, infused with, or otherwise carries a composition that comprises a polynucleotide or vector as described herein for Bmx gene therapy.

### Kits

As an additional aspect, the invention includes kits which comprise one or more compounds or compositions of the invention packaged in a manner which facilitates their use to practice methods of the invention. In a simplest embodiment, such a kit includes a compound or composition described herein as useful for practice of a method of the invention (e.g., gene therapy vectors containing Bmx polynucleotides for administration to a patient), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration or for practicing a screening assay.

Additional aspects and details of the invention will be apparent from the following examples, which are intended to be illustrative rather than limiting.

### EXAMPLES

Additional aspects and details of the invention will be apparent from the following examples, which are intended to be illustrative rather than limiting. Example 1 describes the levels of Bmx tyrosine kinase in ischemic hindlimb. Example 2 describes the generation of Bmx-SK transgenic mice. Example 3 describes Bmx-SK-Tg augmentation of perfusion recovery in the ischemic injured animals. Example 4 describes post-ischemic arteriogenesis and angiogenesis in Bmx-SK-Tg and Bmx-KO mice. Example 5 describes gene expression of proangiogenic factors in Bmx-SK-Tg mice in response to ischemia. Example 6 describes the role of Bmx in mobilization of bone marrow-derived EPC. Example 7 describes administration of a Bmx gene therapy vector.

### Example 1

### Bmx is Highly Induced and Activated in Ischemic Hindlimbs

To determine the role of Bmx in inflammatory arteriogenesis/angiogenesis, Bmx expression and activation was examined in response to ischemic injury in mouse hindlimbs by surgical arteriectomy of the left femoral artery.

In the animal studies, 8-12 week old male C57BL/6 mice (Jackson Laboratories, Bar Harbor, Me.) were used for all experiments. Mouse ischemic hindlimb model was performed as described previously [Bauer et al., Proc Natl Acad Sci USA (2005) 102:204-209; Yu et al., Proc Natl Acad Sci USA (2005) 102:10999-11004]. Briefly, following anesthesia (79.5 mg/kg ketamine, 9.1 mg/kg; xylazine), the left femoral artery was exposed under a dissection microscope. The proximal end of the femoral artery and the distal portion of the saphenous artery were ligated. All branches between these two sites were ligated or cauterized, and arteriectomy was performed. A sham operation was performed as a control, lacking femoral artery ligation but comprising the skin incision. The upper and lower limbs of the ischemic (left legs) and non-ischemic (right legs) were harvested on days 3, 7, 14 and 28 post-surgery, and Bmx protein was determined by Western blot with anti-Bmx antibody (Santa Cruz Biotechnology, Santa Cruz, Calif.). Bmx activity can be measured in various EC (e.g., human umbilical vascular endothelial cells (HUVEC), Bovine Aortic Endothelial Cells (BAEC) or murine lung endothelial cells (MLEC)).

For the Western blot assay, EC from control and treated mice were isolated at the designated day post surgery, washed twice with cold PBS and lysed in 1.5 ml of cold lysis buffer [50 mM Tris-HCI, pH 7.6, 150 mM NaCI, 0.1% Triton X-100, 0.75% Brij 96 buffer (oleyl alcohol EO (10)), 1 mM sodium orthovanadate, 1 mM sodium fluoride, 1 mM sodium pyrophosphate, 10 μg/ml aprotinin, 10 μg/ml leupeptin, 2 mM PMSF, 1 mM EDTA] for 20 min on ice. Protein concentrations were determined with a Bio-Rad kit. The cell lysates were subjected to SDS-PAGE followed by immunoblotting (Immobilon P, Millipore, Milford, Mass.) with a specific antibody against Bmx. The chemiluminescence was detected using an ECL kit according to the instructions of the manufacturer (Amersham Life Science, Arlington Heights, Ill.).

Bmx protein levels were highly induced in the ischemic lower hindlimb, and the induction peaked at day 3 but declined to the basal level by day 14. Expression of Bmx in the ischemic upper hindlimb was also weakly induced and persisted up to 4 weeks. In contrast, Bmx was not induced in the non-ischemic hindlimbs. Day 3 samples were then used to determine Bmx mRNA induction by a real-time PCR (qRT-PCR). Consistent with Bmx protein expression, Bmx mRNA was induced in both upper and lower parts of ischemic hindlimb compared to the non-ischemic hindlimb, showing approximately an eight-fold increase in lower hindlimb and approximately a 3 fold increase in the upper hindlimb. To determine if induced Bmx is active, Bmx activation were determined by Western blot with phospho-specific antibody (anti-pY40) (Cell Signaling Technology, Danvers, Mass.) using the same protocol as described above for the anti-Bmx Western blot. Alternatively, Bmx activity can be measured by immunoprecipitating Bmx with anti-Bmx followed by an in vitro kinase assay using a Bmx substrate peptide, e.g. a peptide from PAK1 based on a protocol kinase activity assay (Liu et al., J Clin Invest (2001) 107:917-23; Liu et al., Cir. Res. (2002) 90:1259-66). Briefly, a total of 400 μg cell lysates are immunoprecipitated with 5 μg of antibody against Bmx (Santa Cruz). The immunoprecipitates are mixed with 10 μg GST-PAK1 protein or 1 μg of PAK1 peptide suspended in kinase buffer (20 mM Hepes, pH 7.6, 20 mM MgCl2, 25 mM P-glycerophosphate, 100 μM sodium orthovanadate, 2 mM DTT, 20 μM ATP) containing 1 μl (10 μCi) of [γ-32P] ATP. The kinase assay is performed at 25° C. for 30 min. The reaction is terminated by the addition of Laemmli sample buffer and the products resolved by SDS-PAGE (12%) followed by protein transferring to a membrane (Immobilon P). The phosphorylated substrate is visualized by autoradiography. The membrane is further used for Western blot with anti-Bmx.

Results of the Western blot showed that Bmx phosphorylation was significantly upregulated in the ischemic hindlimbs compared to the non-ischemic tissues. The lower limb showed greater responses in Bmx induction and activation than the upper limb.

To determine the expression of Bmx in the vascular endothelium, sections of vascular endothelium were analyzed by immunohistochemistry with anti-Bmx antibody. Mice were sacrificed at day 3 post-surgery and muscles of the lower limbs were harvested, methanol fixed and paraffin embedded. Tissue sections (5 μm thick) were stained using anti-Bmx antibody, anti-PECAM-1 antibody (Pharmingen, San Diego, Calif.) which can detect circulating platelets, monocytes, neutrophils, some T cells and endothelial cells, and anti-smooth muscle alpha actin (SMA) antibody (Dako, Carpinteria, Calif.) to detect smooth muscle cells. Bound primary antibodies were detected using avidin-biotin-peroxidase (NovaRed™ peroxidase substrate kit, Vector Laboratories, Burlingame, Calif.). Pictures from 4 random areas of each section, and 5 sections per mice were taken using a Kodak digital camera mounted on a light microscope (40× objective).

Results show that Bmx was primarily induced in vascular endothelium including capillaries. As a control, Bmx expression is primarily detected in large arteries in non-ischemic tissues. These results demonstrate that Bmx is upregulated in both lower and upper hindlimbs in ischemic hindlimbs compared to control animals. Additionally, Bmx kinase is phosphorylated indicating the protein is in its activated state during ischemic injury.

### Example 2

### Generation of Bmx-SK Transgenic Mice

The upregulation of Bmx in ischemic hindlimb in Example 1 indicates that Bmx might play a critical role in ischemic-induced tissue repair. The availability of Bmx-KO mice that are developmental “normal” permits the testing of the role of Bmx in post-natal hindlimb angiogenesis and remodeling, despite its relative lack of importance during embryonic vasculogenesis. To compare the Bmx-KO mice, mice expressing a constitutively active form of Bmx were generated.

In order to carry out the studies, Bmx transgenic mice were first generated. A Bmx construct was generated containing the SH2 and the kinase domains (Bmx-SK) (SEQ ID NO: 4). The human Bmx-SK cDNA with a FLAG-tag sequence at 5′-end was inserted into a Tie-2 enhancer/promoter construct at the NotI site between a Tie-2 promoter and enhancer. The plasmid was linearized with SalI digestion and the pronuclear injection (onto C57BL/6 background) was performed at Yale Transgenic Core Facility in New Haven, Conn. The founder transgenics were identified by PCR of tail DNA with a 5′ primer of FLAG and 3′ primer of Bmx-SK.

Because Bmx was primarily induced in vascular endothelium and bone marrow, the transgene was generated expressing the constitutively active Bmx-SK under the control of a Tie-2 enhancer/promoter. A longer Tie-2 enhancer fragment (15 kb) was used rather than a short one (1.6 kb) because it obtains bone marrow and uniform vascular expression, especially in adult tissues [Schlaeger et al., Proc Natl Acad Sci USA. (1997) 94:3058-63]. Two founder mice were obtained as determined by PCR genotyping and successfully bred in the C57BL/6 background to the 10th generation. Bmx-SK mRNA was highly expressed in vascularized tissues including aorta, brain, heart and hindlimb as determined by qRT-PCR. Briefly, total RNA of lower limb muscles was isolated by using phenol/chloroform and isolated using RNeasy kit with DNase I digestion (Qiagen, Valecia, Calif.). Reverse transcription was done by standard procedure (Super Script First-Strand Synthesis System, Qiagen) using 1 μg total RNA. Quantitative real-time PCR was performed by using iQ SYBR Green Supermix on iCycler Real-Time Detection System (Bio-Rad Laboratories, Inc. Hercules, Calif.). Specific primers for VEGF-A, VEGF-B, VEGF-C, Flk-1, Fit-1, Angiopoitein-1 (Ang-1), Angiopoitein-2 (Ang-2), Tie-2, PDGF-A, PDGF-B, PDGF-A receptor, PDGF-β receptor and 18S ribosomal RNA as an internal control were used. Relative amount of mRNA in C57BL/6 and eNOS (−/−) mice lower limb muscle were taken at 3 days and 2 week post-ischemia as controls.

Bmx-SK protein was expressed in hindlimb muscle tissue of Bmx-SK-Tg mice (TG) but not of C57BL/6 mice as determined by Western blot with anti-Flag antibody. Furthermore, Bmx-SK was specifically detected in vessels of hindlimb by immunohistochemistry with anti-Bmx antibody. As controls, a basal level of Bmx was detected in the vasculature of C57BL/6 mice, but not of the Bmx-KO mice. The Bmx-SK-Tg mice showed normal development, gross growth, and breeding.

### Example 3

### Bmx-SK-Tg Augmented Perfusion Recovery in the Ischemic Hindlimbs

In order to determine the effects of a lack of Bmx protein or the expression of constitutively active Bmx on ischemic injury, C57BL/6, Bmx-KO and Bmx-SK-Tg male mice were subjected to femoral artery ligation and lower limb blood flow was measured at days 0, 3, 14, and 28 post surgery.

Blood flow was measured by PeriFlux system with Laser Doppler Perfusion Module (LDPU) unit (Perimed, Inc., North Royalton, Ohio). A deep measurement probe was placed directly on the gastrocnemius muscle to ensure a deep muscle flow measurement. Ischemic and non-ischemic limb perfusion was measured pre-, post-surgery, 3 days, 2 weeks and 4 weeks after surgery. The final blood flow values were expressed as the ratio of ischemic to non-ischemic hind limb perfusion.

To more precisely evaluate the mobility of mice after limb ischemia, we designed a scoring system. 0=normal; 1=pale foot or gait abnormalities; 2=gangrenous tissue in less than half the foot without lower limb necrosis; 3=gangrenous tissue in less than half the foot with lower limb necrosis; 4=gangrenous tissue in greater than half the foot; 5=loss of half lower limb. Clinical outcome of all mice were observed and recorded at the same time points of blood flow measurement.

On day 14 post surgery, Bmx KO mice showed an average clinical score of greater than 3, showing a moderate to severe phenotype. By day 28 post-surgery, Bmx-KO mice showed a severe phenotype compared to C57BL/6 and Bmx-SK-Tg mice (average clinical score of greater than 4) based on the clinical scoring system described above and in [Bauer et al., Proc Natl Acad Sci USA (2005) 102:204-209]. 10 out of 14 Bmx-KO mice had severe necrosis of the feet. To precisely determine functional defects in Bmx-KO mice, blood flow was measured and ischemic and non-ischemic limb perfusion were measured pre- and post-surgery, 3, 14 and 28 days after surgery. Before surgery, the ratio of left leg to right leg gastrocnemius blood flow is 1. Post-surgery, flow dropped to approximately 0.4 ratio by 30 minutes after surgery, and returned to a ratio of 1 over 4 weeks in C57BL/6 mice. Bmx-SK-Tg mice showed a drop to approximately 0.4 at 30 minutes, but demonstrated augmented recovery of hindlimb perfusion and flow returned to normal at 2 weeks post-injury. In contrast, there was a marked impairment in gastrocnemius blood flow in Bmx-KO mice, down to 0.4 by 30 minutes post surgery, and further decreasing to a ratio of 0.2 at day 3. On day 28, the blood flow ratio of Bmx-KO mice was only back to approximately 0.4.

These results suggest that the impairment in blood flow in Bmx-KO is associated with a marked increase in clinical severity.

To further define the functional defects in Bmx-KO mice, skeletal muscle contraction stimulated hyperemia was examined in the gastrocnemius muscle in C57BL/6, Bmx-SK and Bmx-KO mice at baseline and after ischemia. A hyperemic response is a physiological condition resulting from a transient local ischemic event, leading to an increased local blood flow (or vasodilation) to the affected area.

Electrical stimulation was performed as follows. After anesthesia, mice were placed on a heated pad. The adductor muscle group and gastrocnemius muscle were exposed by a middle line incision of the limb. After baseline gastrocnemius blood flow was measured, adductor muscles were stimulated with 2 electrodes at 2 Hz, 5 mA by using electrostimulator for 2 min. Blood flow was taken and recorded by MacLab Chart software (ADInstruments, Colorado Springs, Colo.) during stimulation and for 10 minutes post-stimulation.

Electrical stimulation of the adductor muscle groups in the upper legs of C57BL/6, Bmx-SK or Bmx-KO mice results in a marked increase in peak blood flow measured in the gastrocnemius muscle group of all strains when compare pre-stimulation (at time 0) are compared to peak response at 2 min and recovery phase (when stimulator turned off). The return to baseline flow is slightly delayed in Bmx-KO mice compared to C57BL/6 or Bmx-SK mice.

The same physiological response was measured after limb ischemia in C57BL/6, Bmx-SK and Bmx-KO mice at 2 weeks post-ischemia. Electrical stimulation of the adductor muscle groups in the upper leg of Bmx-SK mice resulted in a similar increase in peak blood flow measured in the gastrocnemius muscle group compared to pre-ischemia. C57BL/6 mice showed a 70% of peak response. However, Bmx-KO showed a markedly diminished peak response (approximately 30%) and the return was significantly delayed compared to pre-ischemia.

These data show that Bmx-SK mice have enhanced hyperemic responses after ischemia, whereas Bmx-KO have reduced hyperemic responses.

### Example 4

### Post-Ischemic Arteriogenesis and Angiogenesis are Enhanced in Bmx-SK-Tg Mice but Impaired in Bmx-KO Mice

Enhanced clinical recovery and limb perfusion could be due to increased arteriogenesis from existing vessels of the upper limb, increased neovascularization/vessel maturation in the lower limb, or/and increased mobilization of endothelial cell progenitors. To determine what factors may be involved in this enhanced recovery, ischemia-initiated arteriogenesis was examined in C57BL/6, Bmx-KO and Bmx-SK-Tg mice by Microfil casting to visualize vascular architecture.

For MICROFIL® perfusion, 4 weeks after femoral ligation, mice were anesthetized and perfused with 20 ml of phosphate buffered saline (PBS) at 37° C. plus 10 units/ml heparin at a flow rate of 10-15 ml/min through the left ventricle. After PBS, mice received 20 ml of 4% paraformaldehyde, and 15 ml of MICROFIL® [MV-112 (white), Flowtech, Carver, Mass.]. The MICROFIL polymerized overnight at 4° C., and the collagen gels and underlying abdominal musculature were harvested and clarified in graded glycerol solutions (40-100% glycerol in water, increased by 20% glycerol at 24-h intervals). The clarified specimens were viewed on an SMZ1000 dissecting microscope (Nikon).

Time course studies in C57BL/6 mice suggest that collateral artery growth in the ischemia hindlimb was clearly visualized at 4 weeks post-ischemia. The collateral growth in C57BL/6, Bmx-KO and Bmx-SK mice was then compared. Results from C57BL/6 mice (WT) after 4 weeks of ischemia, demonstrated an enlargement of gracilis arteries (collateral artery growth) compared to the contralateral leg. Bmx-SK-Tg showed enhanced arterialization as determined by the ratio of diameter in the left arteries vs the right arteries. The ratio of the diameter in WT mice was measured at approximately 1.5 whereas that of Tg mice was increased to 2.0. In contrast, Bmx-KO showed complete lack in enlargement of gracilis arteries, with the ratio of diameter decreasing to approximately 0.75. These data document a critical role of Bmx in ischemic-mediated arteriogenesis.

Secondary to upper limb ischemia, shear stress dependent changes in blood flow will promote capillary angiogenesis in the lower leg. To determine the influenced of Bmx on angiogenesis, the ischemia-responsive regions in the lower limb were characterized by histology. Tissue sections showed that a small triangle area of gastrocnemius muscle was most responsive to ischemia in the increase of capillary formation, macrophage infiltration and myocyte proliferation. This region was the focus to determine ischemia-induced angiogenesis.

After 4 weeks of ischemia, there was an increase in CD31-(PECAM-1)-positive endothelial cells (EC) surrounding the skeletal muscle myocytes in C57BL/6 mice. Quantitation of the number of capillaries and ratio of capillary/fiber showed an increase of capillaries in Bmx-SK mice to approximately 4500 capillaries/mm² compared to approximately 2500 cap/mm² in WT mice. Bmx-KO mice showed only approximately 1600 cap/mm² after ischemia. The ratio of CD31 infiltrating cells to myocytes was also calculated. WT mice demonstrated a ratio of CD31⁺ cells/myocytes of approximately 2.0 while Bmx-SK mice demonstrated a ratio of approximately 2.6 CD31+ cells/myocyte. Bmx KO mice showed levels similar to non-ischemic control mice, of approximately 1.0.

Stable angiogenesis is believed to occur contemporaneously with pericyte recruitment (vessel maturation) mediated by platelet derived growth factor and/or angiopoietin-1. In order to examine the recruitment of pericytes to angiogenic capillary sprouts post-ischemia, thin, serial sections were immunostained with anti-CD31 or anti-smooth muscle a-actin (SMA) antibody. SMA-positive capillaries increased in C57BL/6 after 4 weeks of ischemia to approximately 150 SMA+ cells/mm². Bmx-KO showed reduced numbers of SMA+ cells, down to approximately 125 SMA+ cells/mm² while Bmx-SK Tg mice demonstrated significantly increased numbers of SMA+ cells or approximately 350 SMA+ cells/mm².

These results show that CD31 positive endothelial cells surrounding the skeletal muscle myocytes (neovascularization) and SMA-positive SMC (pericyte recruitment) were significantly increased in Bmx-SK-Tg mice but reduced in Bmx-KO mice compared to C57BL/6 secondary to ischemia as quantified by capillary number/mm², by ratio of capillary/fiber and number of SMA-positive vessel/mm².

### Example 5

### Gene Expression of Proangiogenic Factors is Enhanced in Bmx-SK-Tg Mice in Response to Ischemia

The increase in monocyte and myocyte infiltration in tissue after induction of ischemic injury indicates that other angiogenic factors may be increased as a result of Bmx increase.

In order to analyze if there is an increase in angiogenic molecules at the site of injury, gene expression profiles of several growth factors and their cognate receptors that may contribute to ischemia-induced arteriogenesis/angiogenesis were examined. Because macrophages are inflammatory cells that produce several angiogenic factors and cytokines which in turn activate gene expression in EC and SMC, macrophage and lymphocyte infiltration into ischemia hindlimb (gastrocnemius) was examined by immunostaining with the macrophage-specific anti-F4/80 antibody and anti-CD3 antibody, respectively. No macrophages were detected in non-ischemia limb while ischemia induced drastic macrophage infiltration (˜500 macrophages/mm²) on day 3 post-surgery in Bmx-SK Tg animals. Macrophages were detected on day 7 and declined by day 14 in C57BL/6 mice. similar kinetics were observed for lymphocyte Bmx-KO mice showed 2-fold reduction (to approximately 150 mac/mm²) in macrophage numbers and in T lymphocyte numbers compared to control mice (approximately 350 mac/mm²), whereas Bmx-SK mice had 2-3-fold increase (to approximately 700 mac/mm²) in macrophage infiltration and an increase in T lymphocyte infiltration compared to C57BL/6 mice.

Gene expression of angiogenic factors and pro-inflammatory cytokines as well as their cognate receptors was determined in wild type mice by quantitative RT-PCR at days 0, 3, 7, 14 and 28 days post-surgery. The levels of gene expression were compared in ischemic and contralateral and non-ischemic gastronemius muscle groups. Ischemia rapidly induced gene expression of pro-inflammatory molecules as shown in Table 1.

TABLE 1

| | | Proinflammatory | |
|---|---|---|---|
| Day | | Cytokines | Angiogenic genes |
| post-surgery | TNFR2 | (TNF, VCAM-1) | (Bmx, Ang-2, PIGF) |
| Day 3 | 250 | 25 | 4 |
| Day 7 | 30 | 10 | 3 |
| Day 14 | 2 | 1 | 2 |
| Day 28 | 2 | 1 | 2 |

* Data represent the fold increases of each gene by taking the non-ischemia hindlimb from C57BL/6 as a value of 1.0. All values are approximate values.

The early response genes (pro-inflammatory cytokines and angiogenic genes) peaked at day 3 and declined by day 14. The most drastic induction by ischemia is TNFR2 gene (up to 250-fold). However, the cognate receptors VEGFR-2, Tie-2 and PDGFR-β, primarily expressed on EC or/and SMC/pericytes, showed a similar kinetics of flow recovery in the ischemia hindlimb, probably reflecting the pattern of vessel growth and maturation, decreased to approximately 0.5 fold of normal at day 3 and increased back to baseline levels by day 14. In contrast, expression of some genes including TNFR1 and TRAF2 was not significantly altered. Gene expression of these molecules was compared in Bmx-KO and Bmx-SK mice. Results showed that expression of these angiogenic and proinflammatory genes showed 2-5-fold reduction in Bmx-KO mice but had 1.8-3.5-fold increase in Bmx-SK mice [Tie-2, Ang2 showed 3.5 and 3 fold increase, respectively, all other factors measured (PIGF, VEGFR, PDGFR, TNF, TNFR2, VCAM-1) showed approximately 2.5 fold increase].

The levels and kinetics of gene expression of these angiogenic and proinflammatory genes appeared to be correlated with the number of infiltrated macrophage, consistent with the notion that macrophages may be the cell type producing angiogenic factors and cytokines which in turn may activate gene expression in EC and SMC.

### Example 6

### Role of Bmx in Mobilization of Bone Marrow-Derived EPC

Bmx was initially identified as a bone morrow tyrosine kinase which suggests that the role of bone morrow-derived cells in ischemia-induced angiogenesis should be investigated. To analyze the role of bone-marrow derived cells in ischemia, reciprocal bone morrow transplantation experiments were performed in C57BL/6 mice and Bmx-KO. Bone marrow transplantation (BMT) was performed in lethally irradiated mice (irradiation 2×5.5 Gy within 3 h) using cells derived from donor mice, and bone marrow cells from Bmx-KO mice were harvested by flushing the femur. Red cells were removed by a lysis using ammonium chloride and subsequent washing with PBS. Cells were counted and 1×10⁵ cells were injected into the tail vein of the recipient. Successful BMT was controlled by FACS analysis of peripheral blood six weeks after BMT.

First, C57BL/6 mice were subjected to ligation and subsequently received lethal irradiation followed by transplantation of bone morrow cells from Bmx-KO mice. Results showed that no statistically significant difference was observed between both groups before and immediately after femoral artery ligation. Starting three days after operation, blood flow recovery in transplanted mice was significantly impaired compared to wild types (0.38±0.08 {control} vs. 0.18±0.01 {transplanted mice}, p<0.05), day 7 (0.6±0.09 vs. 0.36±0.03, p<0.05) and day 14 (0.8±0.11 vs. 0.48±0.06. p<0.05). No statistically significant difference was observed at day 21 between two groups. These data suggest that Bmx in bone morrow-derived cells is critical for the early phase of ischemia-induced tissue repair.

Bone-morrow-derived endothelium progenitor cells (EPC) have been implicated in ischemia-induced angiogenesis and flow recovery. to determine the role of Bmx in EPC migration, deficiency of Bmx in Bmx-KO or overexpression of Bmx in Bmx-SK-Tg mice after ischemia-mediated peripheral EPC mobilization was assessed.

For the endothelium progenitor cell (EPC) mobilization assay, after anesthesia and heparinization, blood was drawn by cardiac puncture. Mononuclear cells were isolated by a density gradient method using Histopaque-1077 (Sigma). 500 μl blood was mixed with 2 ml PBS, gently added to 2 ml Histopaque-1077 and centrifuged at 400 g for 30 minutes. The mononuclear fraction was collected, washed in PBS and following red cell lysis with Ammonium Chloride solution (StemCell Technologies), 1×10⁶ cell/cm² were seeded on fibronectin coated slides (Clontech, San Jose, Calif.). Cells were allowed to differentiate in EGM-2 SingleQuots medium (Cambrex, East Rutherford, New Jersey) containing VEGF-A, FGF, IGF-1, Hydrocortisone, Ascorbic acid, GA 1000, Heparin, 1% Gentamicin/Streptomycin (GIBCO, Carlsbad, Calif.) and 5% FBS. Medium was changed every other day. After 5 days of in vitro culture, cells were incubated in 10 μg/ml acethylated low density lipoprotein (Ac-Dil-LDL) (Biomedical Technologies) for 4 hours and Ac-Dil-LDL positive cells were photographed and counted in ten low power (10×) fields for each animal.

Limb ischemia triggered a 5-fold increase of EPC mobilization in C57BL/6 mice over baseline levels. EPC mobilization in response to ischemia was reduced by 3-fold in Bmx-KO mice compared to wild type ischemic mice, but showed a 2.5-fold increase in Bmx-SK-Tg mice compared to wild type ischemic mice.

To determine the effects of Bmx on EPC function, VEGFR-2 signaling, which is critical for EPC differentiation to EC [Takahashi et al., Nat Med (1999) 5:434-38. Asahara et al., Embo J (1999) 18:3964-72; Aicher et al., Nat Med 2003) 9:1370-76], was examined. It was first determined if Bmx and Bmx-SK are expressed in isolated EPC. Expression of Bmx was detected in EPC derived from C57BL/6 and Bmx-SK-Tg, but not those from Bmx-KO mice, as determined by Western blot with anti-Bmx antibody. Bmx-SK was also detected in bone marrow and bone marrow-derived EPC from Bmx-SK-Tg mice, consistent with the fact that Tie-2 enhancer/promoter functions in bone marrow (Schlaeger et al., supra). EPCs isolated from Bmx-SK-Tg show increased VEGFR2 expression and phosphorylation in response to VEGF compared to those from C57BL/6 mice. In contrast, EPCs isolated from Bmx-KO mice show a blunted increase in VEGFR2 expression and phosphorylation in response to VEGF compared to those from C57BL/6 mice. These data suggest that Bmx functions as a positive regulator of VEGFR2 signaling which is involved in EPC mobilization.

The data presented above demonstrate that Bmx in bone marrow-derived cells plays a critical role in ischemia-induced vascular remodeling. This is supported by the results that bone marrow-derived cells from Bmx-KO implanted to C57BL/6 mice impaired blood flow recovery compared to the WT mice. Mobilization of EPC from bone marrow to peripheral blood and injury sites has been proposed to account for the function of bone marrow cells. The results show that EPC mobilization is significantly reduced in Bmx-KO mice but is dramatically augmented in the Bmx-SK-Tg mice.

Collectively, this data strongly support the role of Bmx in mediating in ischemia-induced arteriogenesis and angiogenesis in vivo through multiple pathways by enhancing collateral growth, EPC mobilization and angiogenesis, resulting in enhanced recovery of blood flow and vascular remodeling. These results suggest that Bmx may be a novel target for the treatment of vascular diseases such as coronary artery and peripheral arterial diseases.

### Example 7

### Bmx Gene Therapy

As demonstrated above, Bmx plays a critical role in arteriogeneis and angiogenesis in ischemic injury. To demonstrate the efficacy of Bmx as a gene therapy agent, ischemic mice as described above are administered a Bmx gene therapy vector and the extent of angiogenesis and arteriogenesis is measured.

Wild type, Bmx-KO and Bmx-SK-Tg mice are induced with hindlimb ischemia as described above. The subject animals may be administered Bmx gene therapy vector before injury (e.g., up two weeks before injury) as described in Lan et al [J Endovasc Ther (2005) 12:469-78] in a rat model, in Emanueli et al., [Diabetes (2004) 53:1096-103] in a mouse diabetes model, or after injury as described in Dai et al., [Circulation (2004) 110:2467-75] in a rabbit ischemia model. Administration of the vector may be performed at a pre-determined time, such as 6 weeks before ischemic injury, two weeks before, 7 days before, 3 days before or at the same time as injury. Similarly, gene therapy may be administered at an appropriate time after injury such as a few hours, 1 day, 3 days after, 7 days after, or 2 weeks after injury. A single dose and repetitive dosing are assayed

In different experiments, the gene therapy vector is a viral vector, a plasmid vector, or any other vector vehicle described herein or known in the art. The Bmx gene therapy vector is administered over a range of concentrations and by any of the routes of administration described herein.

Angiogenic factors and extent of angiogenesis and arteriogenesis are measured in animals receiving Bmx gene therapy as described above. An increase in angiogenic and arteriogenic characteristics, such as increased vessel growth, increased vessel diameter, improved blood flows, and the like, compared to control mice indicates that Bmx gene therapy is effective at improving impaired vasculature as a result of ischemia.

Gene therapy is also carried out in the ischemic hindlimb injury model above using an ex vivo transduction model, wherein cells are removed from the subject or a cell-line representative of cells removed from the subject are transduced with a Bmx gene therapy vector. Endothelial cells, endothelial progenitor cells or bone marrow derived cells are transfected ex vivo with the Bmx transgene, and the transfected cells are administered to the mammalian subject. Especially in the case of progenitor cells, the donor and recipient animals may be different.

As an exemplary protocol, wild type, Bmx KO and Bmx-SK-Tg mice are administered an ischemic injury as described above. Endothelial progenitor cells are removed from congenic animals or a mouse endothelial cell line is used for the transduction. In one aspect, endothelial progenitor cell culture is carried out as described in Iwaguro et al., (Circulation (2002) 105:732-38) for human EPCs. Briefly, peripheral blood mononuclear cells from animals are plated on fibronectin-coated (Sigma) culture dishes and maintained in EC basal medium-2 (EBM-2) (Clonetics) supplemented with 5% fetal bovine serum, VEGF-A, fibroblast growth factor-2, epidermal growth factor, insulin-like growth factor-1, and ascorbic acid. After 4 days in culture, nonadherent cells are removed by washing, new media is applied, and the culture maintained. After 7 days in culture, cells are transduced with an adenovirus, or other viral vector, encoding the Bmx gene or control gene. To establish optimum conditions for EPC viral gene transfer serum concentration, virus incubation time and virus concentration are evaluated. Preliminary experiments are performed to determine the optimal transfection protocol, but an exemplary protocol includes EPCs transduced with 1000 MOI Ad/Bmx or Ad/Bmx-SK or control for 3 hours in 1% serum media. After transduction, cells are washed with PBS and incubated with EPC media for 24 hours before transplantation.

Angiogenic factors and extent of angiogenesis and arteriogenesis is measured in animals receiving Bmx gene therapy as described above. An increase in angiogenic and arteriogenic characteristics, such as increased vessel growth, increased vessel diameter, increased blood flows, and the like compared to control mice indicates that Bmx gene therapy is effective at improving impaired vasculature as a result of ischemia.

An increase in therapeutic angiogenesis as a result of Bmx gene therapy in mice also provides a promising therapy for human patients suffering from impaired vascular structure due to ischemic injury or other impairment of vascular function, such as diabetes.

Numerous modifications and variations in the invention as set forth in the above illustrative examples are expected to occur to those skilled in the art. Consequently only such limitations as appear in the appended claims should be placed on the invention.

The references cited herein throughout, to the extent that they provide exemplary procedural or other details supplementary to those set forth herein, are all specifically incorporated herein by reference.

What is claimed:

1. A method for increasing arteriogenesis or angiogenesis in a mammalian subject comprising:
administering to a mammalian subject in need of arteriogenesis or angiogenesis a composition that comprises a polynucleotide that comprises a nucleotide sequence that encodes a Bmx tyrosine kinase amino acid sequence selected from the group consisting of:
(a) the amino acid sequence set forth in SEQ ID NO: 2;
(b) the amino acid sequence set forth in SEQ ID NO: 4;
(c) fragments of (a) or (b) that retain BMX tyrosine kinase activity; and
(d) amino acid sequences that are at least 70% identical to (a), (b), or (c) and that retain BMX tyrosine kinase activity.

2. A method for increasing arteriogenesis or angiogenesis in a mammalian subject comprising:
administering to a mammalian subject in need of arteriogenesis or angiogenesis a composition that comprises cells transduced with a polynucleotide that causes elevated expression of a BMX tyrosine kinase amino acid sequence selected from the group consisting of:
(a) the amino acid sequence set forth in SEQ ID NO: 2;
(b) the amino acid sequence set forth in SEQ ID NO: 4;
(c) fragments of (a) or (b) that retain BMX tyrosine kinase activity; and
(d) amino acid sequences that are at least 70% identical to (a), (b), or (c) and that retain BMX tyrosine kinase activity.

3. The method of claim 2, wherein the polynucleotide encodes the polypeptide.

4. The method of any one of claims 2-3, wherein the cells are selected from the group consisting of endothelial cells, endothelial precursor cells, Bone marrow derived cells including monocytes, macrophages, and stem cells with the potential to differentiate into endothelial cells.

5. The method of any one of claims 1-4, wherein the polypeptide comprises the amino acid sequence set forth in SEQ ID NO: 2 or 4.

6. The method of any one of claims 1-5, wherein the composition comprises a vector that contains a transgene that includes the polynucleotide operably linked to at least one expression control sequence that promotes expression of the polynucleotide in mammalian cells.

7. The method of claim 6, wherein the at least one expression control sequence is selected from the group consisting of promoters, enhancers, introns, 3′UTR sequences, zinc-finger constructs, and polyadenylation signal sequences.

8. The method of claim 7, wherein the expression control sequence is a promoter selected from the group consisting of: CMV promoter, β-actin promoter, Tie promoter, Tie-2 promoter, VE-cadherin promoter, Endothelial cell specific promoters, bone-marrow specific promoter, and an inducible Tet promoter.

9. The method of any one of claims 6-8, wherein the vector is selected from the group consisting of an adenovirus, an adeno-associated virus, a lentivirus, a plasmid, and a liposome.

10. The method of claim 9, wherein the vector is a replication-deficient virus.

11. The method of any one of claims 1-10, wherein the composition is administered to the subject locally to a site in need of arteriogenesis or angiogenesis.

12. The method of any one of claims 1-11, wherein the composition is administered via a catheter, a membrane, a collar, or a syringe.

13. The method of any one of claims 1-11, further comprising administering the composition in combination with a drug-eluting stent.

14. The method of any one of claims 1-13, wherein the composition is administered to a lumen wall of a blood vessel.

15. The method of any one of claims 1-14, wherein the subject is suffering from a disease or condition associated with impaired circulation.

16. The method of claim 15, wherein the disease or condition is selected from the group consisting of coronary artery disease, peripheral arterial disease, ischemic injury, arterial stenosis, cerebrovascular disease, and renal arterial stenosis.

17. The method of claim 16, wherein the arterial stenosis is selected from the group consisting of atherosclerosis, stenosis of the heart and leg muscles, and stenosis of diabetic patients.

18. The method according to any one of claims 1-17, wherein the composition further includes a pharmaceutically acceptable carrier.

19. The method according to any one of claims 1-18, further comprising administering to the subject a second agent.

20. The method according to any one of claims 1-18, further comprising administering to the subject a cell-permeable peptide.

21. The use of a polynucleotide that encodes a polypeptide with Bmx tyrosine kinase activity for the manufacture of a medicament to treat coronary artery disease or peripheral artery disease, wherein the polypeptide comprises an amino acid sequence selected from the group consisting of:
(a) the amino acid sequence set forth in SEQ ID NO: 2;
(b) the amino acid sequence set forth in SEQ ID NO: 4;
(c) fragments of (a) or (b) that retain BMX tyrosine kinase activity; and
(d) amino acid sequences that are at least 70% identical to (a), (b), or (c) and that retain Bmx tyrosine kinase activity.
